(19) United States
(12) Patent Application Publication
(10) Pub. No.: US 2024/0016544 A1
(43) Pub. Date: Jan. 18, 2024

(54) EMITTER SELECTION BASED ON RADIOPAQUE EMITTER STATIONS FOR INTRAVASCULAR LITHOTRIPSY DEVICE

(71) Applicant: Bolt Medical, Inc., Carlsbad, CA (US)

(72) Inventors: Eric Schultheis, San Clemente, CA (US); Alvin Salinas, San Marcos, CA (US)

(21) Appl. No.: 18/339,901

(22) Filed: Jun. 22, 2023

Related U.S. Application Data
(60) Provisional application No. 63/390,102, filed on Jul. 18, 2022.

(51) Int. Cl. A61B 18/24; A61B 18/26; A61B 18/04

(52) U.S. Cl. CPC A61B 18/24; A61B 18/26; A61B 18/042; A61B 2018/0022

(57) ABSTRACT

A catheter system (100) for placement within a blood vessel (108) having a vessel wall (108A) can be used for treating a treatment site (106) within or adjacent to the vessel wall (108A). The catheter system (100) includes an energy source (124), a plurality of energy guides (122A), and a plurality of emitters (135). The energy source (124) generates energy. Each of the energy guides (122A) is configured to selectively receive the energy from the energy source (124). Each of the energy guides (122A) includes a corresponding guide distal end (122D). The energy that is received by each of the energy guides (122A) is emitted from the corresponding guide distal end (122D). Each of the emitters (135) is positionable near the treatment site (106). Each of the emitters (135) includes the corresponding guide distal end (122D) of one of the energy guides (122A). At least one of the emitters (135) includes a radiopaque material.

### RELATED APPLICATION

This Application is related to and claims priority on U.S. Provisional Patent Application Ser. No. 63/390,102 filed on Jul. 18, 2022, and entitled “EMITTER SELECTION BASED ON RADIOPAQUE EMITTER STATIONS FOR INTRAVASCULAR LITHOTRIPSY DEVICE”. To the extent permissible, the contents of U.S. Application Ser. No. 63/390,102 are incorporated in their entirety herein by reference.

### BACKGROUND

Vascular lesions within vessels in the body can be associated with an increased risk for major adverse events, such as myocardial infarction, embolism, deep vein thrombosis, stroke, and the like. Severe vascular lesions can be difficult to treat and achieve patency for a physician in a clinical setting.

Vascular lesions may be treated using interventions such as drug therapy, balloon angioplasty, atherectomy, stent placement, vascular graft bypass, to name a few. Such interventions may not always be ideal or may require subsequent treatment to address the lesion.

### SUMMARY

The present invention is directed toward a catheter system for placement within a blood vessel having a vessel wall. The catheter system can be used by an operator for treating a treatment site within or adjacent to the vessel wall. In various embodiments, the catheter system includes an energy source, a plurality of energy guides, and a plurality of emitters. The energy source generates energy. Each of the plurality of energy guides is configured to selectively receive the energy from the energy source. Each of the plurality of energy guides includes a corresponding guide distal end. The energy that is received by each of the plurality of energy guides is emitted from the corresponding guide distal end. Each of the plurality of emitters is positionable near the treatment site. Each of the plurality of emitters includes the corresponding guide distal end of one of the plurality of energy guides. At least one of the emitters includes a radiopaque material.

In many embodiments, the radiopaque material is visible when used with fluoroscopy during use of the catheter system in an intravascular lithotripsy procedure.

In some embodiments, the catheter system further includes a catheter shaft and a balloon that is coupled to the catheter shaft. The balloon includes a balloon wall that defines a balloon interior. The balloon is configured to retain a catheter fluid within the balloon interior. The energy guides are disposed along the catheter shaft. The corresponding guide distal end of each of the energy guides is positioned within the balloon interior so that each of the emitters is positioned within the balloon interior.

In certain embodiments, each emitter further includes a corresponding plasma generator that is positioned near the corresponding guide distal end of the one of the plurality of energy guides. The energy that is received by each of the plurality of energy guides is emitted from the corresponding guide distal end and impinges on the corresponding plasma generator so that plasma is generated in the catheter fluid retained within the balloon interior.

In some embodiments, the plasma generation causes bubble formation that generates a pressure wave that imparts pressure adjacent to the vessel wall.

In certain embodiments, the catheter system further includes a plurality of emitter stations that are positioned within the balloon interior. Each emitter station can be positioned at a different longitudinal position within the balloon interior relative to a length of the balloon than each of the other emitter stations. Each emitter station includes at least one of the plurality of emitters. At least one of the plurality of emitter stations includes a radiopaque material.

In many embodiments, each of the plurality of emitter stations includes a radiopaque material that is visible when used with fluoroscopy during use of the catheter system in an intravascular lithotripsy procedure.

In some embodiments, the plurality of emitter stations includes a first emitter station including a first plurality of emitters that are each positioned at a first longitudinal position within the balloon interior, and a second emitter station that includes a second plurality of emitters that are each positioned at a second longitudinal position within the balloon interior that is different than the first longitudinal position.

In certain embodiments, the catheter system further includes a system controller including a processor that controls the energy source so that the energy from the energy source is selectively directed to each of the emitters in any desired pattern of firing.

In some embodiments, the system controller is configured to one of specifically select and specifically deselect the emitters to be activated during use of the catheter system in an intravascular lithotripsy procedure based at least in part on proximity of the emitters to the treatment site.

In certain embodiments, the system controller is configured to selectively activate only those emitters that are positioned most proximate to the treatment site.

In other embodiments, the system controller is configured to selectively deactivate those emitters that are positioned least proximate to the treatment site.

In some embodiments, the catheter system further includes a graphical user interface that includes a plurality of emitter activators that can be used to one of specifically select and specifically deselect the emitters to be activated during use of the catheter system in the intravascular lithotripsy procedure.

In many embodiments, the catheter system further includes a multiplexer that receives the energy from the energy source and directs the energy from the energy source in the form of individual guide beams to each of the energy guides.

In many embodiments, the energy source is a light source that generates pulses of light energy.

In some embodiments, the light source is a laser.

In certain embodiments, each of the plurality of energy guides includes an optical fiber.

The present invention is further directed toward a method for treating a treatment site within or adjacent to a vessel wall, the method including the steps of generating energy with an energy source; selectively receiving the energy from the energy source with each of a plurality of energy guides, each of the plurality of energy guides including a corresponding guide distal end, the energy that is received by each of the plurality of energy guides being emitted from the corresponding guide distal end; and positioning a plurality of emitters near the treatment site, each emitter including the corresponding guide distal end of one of the plurality of energy guides, at least one of the emitters including a radiopaque material.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic cross-sectional view illustration of an embodiment of a catheter system in accordance with various embodiments, the catheter system including a plurality of energy guides and a multiplexer;

FIG. 2A is a simplified schematic top view illustration of a portion of an embodiment of the catheter system including an embodiment of the multiplexer;

FIG. 2B is a simplified schematic perspective view illustration of a portion of the catheter system and the multiplexer illustrated in FIG. 2A;

FIG. 3A is a simplified schematic top view illustration of a portion of an embodiment of the catheter system including another embodiment of the multiplexer;

FIG. 3B is a simplified schematic perspective view illustration of a portion of the catheter system and the multiplexer illustrated in FIG. 3A;

FIG. 4 is a simplified schematic top view illustration of a portion of the catheter system and still another embodiment of the multiplexer;

FIG. 5 is a simplified schematic top view illustration of a portion of the catheter system and yet another embodiment of the multiplexer;

FIG. 6 is a simplified schematic top view illustration of a portion of the catheter system and still another embodiment of the multiplexer;

FIG. 7 is a simplified schematic top view illustration of a portion of the catheter system and still yet another embodiment of the multiplexer;

FIG. 8 is a simplified schematic side view illustration of a portion of an embodiment of the catheter system having features of the present invention, the catheter system including a balloon having a balloon wall that defines a balloon interior, and two emitter stations that are positioned within the balloon interior of the balloon;

FIG. 9 is a simplified schematic view illustration of a portion of another embodiment of the catheter system, the catheter system including four emitter stations that are positioned within the balloon interior of the balloon;

FIG. 10 is a simplified schematic view illustration of a portion of still another embodiment of the catheter system, the catheter system including five emitter stations that are positioned within the balloon interior of the balloon;

FIG. 11A is a fluoroscopic image of a portion of an embodiment of a catheter system that is positioned substantially adjacent to a vascular lesion, the catheter system including four emitter stations that are positioned within the balloon interior of the balloon, the balloon being in an inflated state;

FIG. 11B is a fluoroscopic image of the catheter system illustrated in FIG. 11A that is positioned substantially adjacent to a vascular lesion, the balloon being in a deflated state;

FIG. 12 is a fluoroscopic image of a portion of another embodiment of the catheter system that is positioned substantially adjacent to a vascular lesion; and

FIG. 13 is a simplified illustration of an embodiment of a graphical user interface that is usable as part of the catheter system.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

In various embodiments, the catheter systems and related methods disclosed herein can include a catheter configured to advance to a vascular lesion, such as a calcified vascular lesion or a fibrous vascular lesion, at a treatment site located within or adjacent a blood vessel within a body of a patient. As used herein, the terms “treatment site”, “intravascular lesion” and “vascular lesion” are used interchangeably unless otherwise noted. As such, the intravascular lesions and/or the vascular lesions are sometimes referred to herein simply as “lesions”.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

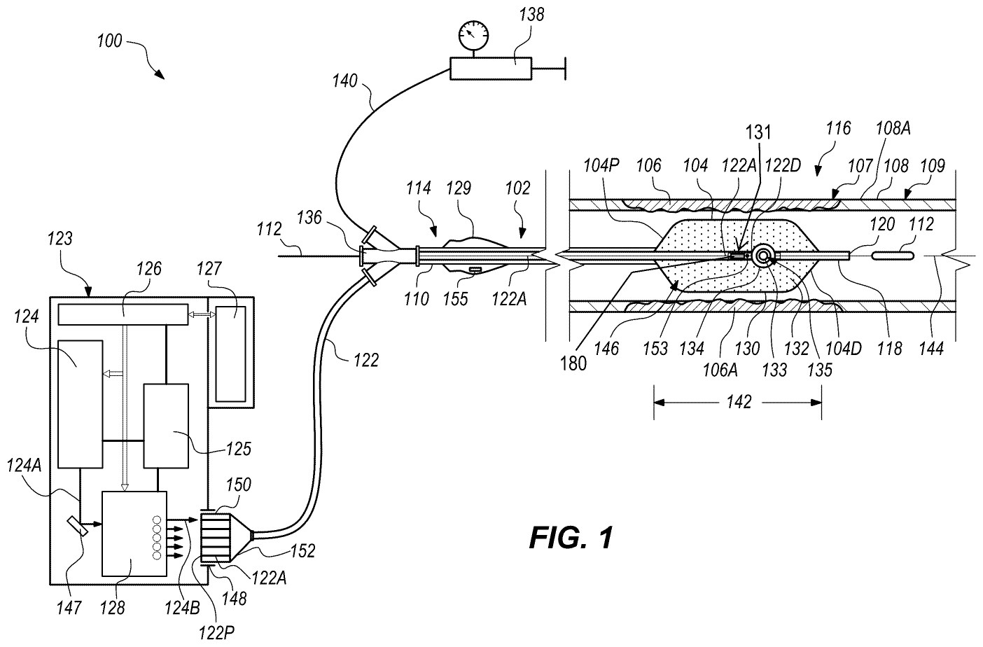

The catheter systems disclosed herein can include many different forms. Referring now to FIG. 1, a simplified schematic cross-sectional view illustration is shown of a catheter system 100 in accordance with various embodiments. The catheter system 100 is suitable for imparting pressure waves to induce fractures in one or more vascular lesions within or adjacent to a vessel wall of a blood vessel or on or adjacent to a heart valve within a body of a patient. In the embodiment illustrated in FIG. 1, the catheter system 100 can include one or more of a catheter 102, an energy guide bundle 122 including one or more energy guides 122A, a source manifold 136, a fluid pump 138, a system console 123 including one or more of an energy source 124, a power source 125, a system controller 126, a graphic user interface 127 (a “GUI”) and a multiplexer 128, a handle assembly 129, and an energy emitting system 131 (also referred to herein as an “emitter system”) including one or more emitter stations 180. Alternatively, the catheter system 100 can include more components or fewer components than those specifically illustrated and described in relation to FIG. 1.

The catheter 102 is configured to move to the treatment site 106 within or adjacent to a vessel wall 108A of a blood vessel 108 within a body 107 of a patient 109. The treatment site 106 can include one or more vascular lesions 106A such as calcified vascular lesions, for example. Additionally, or in the alternative, the treatment site 106 can include vascular lesions 106A such as fibrous vascular lesions. Still alternatively, in some implementations, the catheter 102 can be used at a treatment site 106 within or adjacent to a heart valve within the body 107 of the patient 109.

The catheter 102 can include an inflatable balloon 104 (sometimes referred to herein as a “balloon”), a catheter shaft 110, and a guidewire 112. The balloon 104 can be coupled to the catheter shaft 110. The balloon 104 can include a balloon proximal end 104P and a balloon distal end 104D. The catheter shaft 110 can extend from a proximal portion 114 of the catheter system 100 to a distal portion 116 of the catheter system 100. The catheter shaft 110 can include a longitudinal axis 144. The catheter 102 and/or the catheter shaft 110 can also include a guidewire lumen 118 which is configured to move over the guidewire 112. As utilized herein, the guidewire lumen 118 defines a conduit through which the guidewire 112 extends. The catheter shaft 110 can further include an inflation lumen (not shown) and/or various other lumens for various other purposes. In some embodiments, the catheter 102 can have a distal end opening 120 and can accommodate and be tracked over the guidewire 112 as the catheter 102 is moved and positioned at or near the treatment site 106. In some embodiments, the balloon proximal end 104P can be coupled to the catheter shaft 110, and the balloon distal end 104D can be coupled to the guidewire lumen 118.

The balloon 104 includes a balloon wall 130 that defines a balloon interior 146. The balloon 104 can be selectively inflated with a catheter fluid 132 to expand from a deflated state suitable for advancing the catheter 102 through a patient's vasculature, to an inflated state (as shown in FIG. 1) suitable for anchoring the catheter 102 in position relative to the treatment site 106. Stated in another manner, when the balloon 104 is in the inflated state, the balloon wall 130 of the balloon 104 is configured to be positioned substantially adjacent to the treatment site 106. It is appreciated that although FIG. 1 illustrates the balloon wall 130 of the balloon 104 being shown spaced apart from the treatment site 106 of the blood vessel 108 when in the inflated state, this is done for ease of illustration. It is recognized that the balloon wall 130 of the balloon 104 will typically be substantially directly adjacent to and/or abutting the treatment site 106 when the balloon 104 is in the inflated state.

As an overview, each of the emitter stations 180 of the emitter system 131 and/or the catheter system 100 can include one or more emitters 135 that are configured to generate plasma and/or pressure waves in the catheter fluid 132 within the balloon interior 146. Each of the emitters 135 includes a corresponding guide distal end 122D (also sometimes referred to herein simply as “guide distal end”) of one of the energy guides 122A, which is positioned within the balloon interior 146, and a corresponding plasma generating structure 133 (also referred to herein as a “plasma generator”) that is positioned near, but typically spaced apart from, the guide distal end 122D. Energy from the energy source 124 is directed toward and received by the energy guide 122A, is guided through the energy guide 122A, and is then emitted from the guide distal end 122D of the energy guide 122A. The energy emitted from the guide distal end 122D is directed toward and impinges on and energizes the corresponding plasma generator 133 for purposes of generating the plasma in the catheter fluid 132 within the balloon interior 146.

In various embodiments, the emitter stations 180 and/or the individual emitters 135 can be formed from and/or can include a radiopaque material that is easily visible when used with fluoroscopy during an intravascular lithotripsy procedure. The visibility of the emitter stations 180 and/or the emitters 135 through use of the radiopaque material enables the user or operator to more precisely position the emitter stations 180 and/or the emitters 135 as desired substantially adjacent to the vascular lesions 106A, and/or to selectively activate only those emitter stations 180 and/or emitters 135 that are positioned most proximate to the vascular lesions 106A in order to more effectively disrupt the vascular lesions 106A. By positioning the emitter stations 180 and/or the emitters 135 more precisely substantially adjacent to the vascular lesions 106A at the treatment site 106, and by activating only certain emitter stations and/or emitters based on proximity to the vascular lesions 106A at the treatment site 106, the user or operator can operate the catheter system 100 more effectively and efficiently. Thus, the user and operator can realize savings in money and resources.

The balloon 104 suitable for use in the catheter system 100 includes those that can be passed through the vasculature of a patient 109 when in the deflated state. In some embodiments, the balloons 104 are made from silicone. In other embodiments, the balloon 104 can be made from materials such as polydimethylsiloxane (PDMS), polyurethane, polymers such as PEBAX™ material, nylon, or any other suitable material.

The balloon 104 can have any suitable diameter (in the inflated state). In various embodiments, the balloon 104 can have a diameter (in the inflated state) ranging from less than one millimeter (mm) up to 25 mm. In some embodiments, the balloon 104 can have a diameter (in the inflated state) ranging from at least 1.5 mm up to 14 mm. In some embodiments, the balloon 104 can have a diameter (in the inflated state) ranging from at least two mm up to five mm.

In some embodiments, the balloon 104 can have a length 142 ranging from at least three mm to 300 mm. More particularly, in some embodiments, the balloon 104 can have a length 142 ranging from at least eight mm to 200 mm. It is appreciated that a balloon 104 having a relatively longer length can be positioned adjacent to larger treatment sites 106, and, thus, may be usable for imparting pressure waves onto and inducing fractures in larger vascular lesions 106A or multiple vascular lesions 106A at precise locations within the treatment site 106. It is further appreciated that a longer balloon 104 can also be positioned adjacent to multiple treatment sites 106 at any one given time.

The balloon 104 can be inflated to inflation pressures of between approximately one atmosphere (atm) and 70 atm. In some embodiments, the balloon 104 can be inflated to inflation pressures of from at least 20 atm to 60 atm. In other embodiments, the balloon 104 can be inflated to inflation pressures of from at least six atm to 20 atm. In still other embodiments, the balloon 104 can be inflated to inflation pressures of from at least three atm to 20 atm. In yet other embodiments, the balloon 104 can be inflated to inflation pressures of from at least two atm to ten atm.

The balloon 104 can have various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape. In some embodiments, the balloon 104 can include a drug eluting coating or a drug eluting stent structure. The drug eluting coating or drug eluting stent can include one or more therapeutic agents including anti-inflammatory agents, anti-neoplastic agents, anti-angiogenic agents, and the like.

The catheter fluid 132 can be a liquid or a gas. Some examples of the catheter fluid 132 suitable for use can include, but are not limited to one or more of water, saline, contrast medium, fluorocarbons, perfluorocarbons, gases, such as carbon dioxide, or any other suitable catheter fluid 132. In some embodiments, the catheter fluid 132 can be used as a base inflation fluid. In some embodiments, the catheter fluid 132 can include a mixture of saline to contrast medium in a volume ratio of approximately 50:50. In other embodiments, the catheter fluid 132 can include a mixture of saline to contrast medium in a volume ratio of approximately 25:75. In still other embodiments, the catheter fluid 132 can include a mixture of saline to contrast medium in a volume ratio of approximately 75:25. However, it is understood that any suitable ratio of saline to contrast medium can be used. The catheter fluid 132 can be tailored on the basis of composition, viscosity, and the like so that the rate of travel of the pressure waves are appropriately manipulated. In certain embodiments, the catheter fluids 132 suitable for use are biocompatible. A volume of catheter fluid 132 can be tailored by the chosen energy source 124 and the type of catheter fluid 132 used.

In some embodiments, the contrast agents used in the contrast media can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine-based contrast agents can be used. Suitable non-iodine containing contrast agents can include gadolinium (III)-based contrast agents. Suitable fluorocarbon and perfluorocarbon agents can include, but are not to be limited to, agents such as the perfluorocarbon dodecafluoropentane (DDFP, C5F12).

The catheter fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the catheter fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers usable in the catheter system 100 can include neodymium:yttrium-aluminum-garnet (Nd:YAG−emission maximum=1064 nm) lasers, holmium:YAG (Ho:YAG−emission maximum=2.1 μm) lasers, or erbium:YAG (Er:YAG−emission maximum=2.94 μm) lasers. In some embodiments, the absorptive agents can be water-soluble. In other embodiments, the absorptive agents are not water-soluble. In some embodiments, the absorptive agents used in the catheter fluids 132 can be tailored to match the peak emission of the energy source 124. Various energy sources 124 having emission wavelengths of at least ten nanometers to one millimeter are discussed elsewhere herein.

The catheter shaft 110 of the catheter 102 can be coupled to the plurality of energy guides 122A of the energy guide bundle 122 that are in optical communication with the energy source 124. The energy guide(s) 122A can be disposed along the catheter shaft 110 and within the balloon 104. Each of the energy guides 122A can have a guide distal end 122D that is at any suitable longitudinal position relative to the length 142 of the balloon 104 and/or relative to a length of the guidewire lumen 118. For example, in certain embodiments, a first emitter station 180 can include one or more emitters 135, wherein the guide distal end 122D of each emitter 135 within the first emitter station 180 and a corresponding plasma generator 133, even though they can be slightly spaced apart from one another, can be said to be positioned at a first longitudinal position relative to the length 142 of the balloon 104 and/or relative to a length of the guidewire lumen 118; and a second emitter station 180 can include one or more emitters 135, wherein the guide distal end 122D of each emitter 135 within the second emitter station 180 and the corresponding plasma generator 133, even though they can be slightly spaced apart from one another, can be said to be positioned at a second longitudinal position relative to the length 142 of the balloon 104 and/or relative to the length of the guidewire lumen 118, with the second longitudinal position being different than the first longitudinal position. It is appreciated that the catheter system 100 can include any suitable or desired number of emitter stations 180 that are each positioned at a different longitudinal position relative to the length 142 of the balloon 104 and/or relative to the length of the guidewire lumen 118. It is further appreciated that each emitter station 180 can include any suitable or desired number of emitters 135, with each emitter 135 within a given emitter station 180 necessarily being at approximately the same longitudinal position relative to the length 142 of the balloon 104 and/or relative to the length of the guidewire lumen 118.

In some embodiments, each energy guide 122A can be an optical fiber and the energy source 124 can be a laser. The energy source 124 can be in optical communication with the energy guides 122A at the proximal portion 114 of the catheter system 100. More particularly, as described in detail herein, the energy source 124 can selectively and/or alternatively be in optical communication with each of the energy guides 122A due to the presence and operation of the multiplexer 128.

In some embodiments, the catheter shaft 110 can be coupled to multiple energy guides 122A such as a first energy guide, a second energy guide, a third energy guide, etc., which can be disposed at any suitable positions about and/or relative to the guidewire lumen 118 and/or the catheter shaft 110. For example, in certain non-exclusive embodiments, two energy guides 122A can be spaced apart from one another by approximately 180 degrees about the circumference of the guidewire lumen 118 and/or the catheter shaft 110; three energy guides 122A can be spaced apart from one another by approximately 120 degrees about the circumference of the guidewire lumen 118 and/or the catheter shaft 110; four energy guides 122A can be spaced apart from one another by approximately 90 degrees about the circumference of the guidewire lumen 118 and/or the catheter shaft 110; five energy guides 122A can be spaced apart from one another by approximately 72 degrees about the circumference of the guidewire lumen 118 and/or the catheter shaft 110; six energy guides 122A can be spaced apart from one another by approximately 60 degrees about the circumference of the guidewire lumen 118 and/or the catheter shaft 110; eight energy guides 122A can be spaced apart from one another by approximately 45 degrees about the circumference of the guidewire lumen 118 and/or the catheter shaft 110; or ten energy guides 122A can be spaced apart from one another by approximately 36 degrees about the circumference of the guidewire lumen 118 and/or the catheter shaft 110. Still alternatively, multiple energy guides 122A need not be uniformly spaced apart from one another about the circumference of the guidewire lumen 118 and/or the catheter shaft 110. More particularly, it is further appreciated that the energy guides 122A can be disposed uniformly or non-uniformly about the guidewire lumen 118 and/or the catheter shaft 110 to achieve the desired effect in the desired locations.

In certain embodiments, the guidewire lumen 118 can have a grooved outer surface, with the grooves extending in a generally longitudinal direction along the guidewire lumen 118. In such embodiments, each of the energy guides 122A can be positioned, received and retained within an individual groove formed along and/or into the outer surface of the guidewire lumen 118. Alternatively, the guidewire lumen 118 can be formed without a grooved outer surface, and the position of the energy guides 122A relative to the guidewire lumen 118 can be maintained in another suitable manner.

The catheter system 100 and/or the energy guide bundle 122 can include any number of energy guides 122A in optical communication with the energy source 124 at the proximal portion 114, and with the catheter fluid 132 within the balloon interior 146 of the balloon 104 at the distal portion 116. For example, in some embodiments, the catheter system 100 and/or the energy guide bundle 122 can include from one energy guide 122A to greater than 30 energy guides 122A. The guide distal end 122D of each of the energy guides 122A can be at any suitable or desired longitudinal position within the balloon interior 146 relative to the length 142 of the balloon 104 so as to define any suitable or desired number of emitter stations 180. Alternatively, in other embodiments, the catheter system 100 and/or the energy guide bundle 122 can include greater than 30 energy guides 122A.

The energy guides 122A can have any suitable design that is useful and appropriate for purposes of enabling the generation of plasma and/or pressure waves in the catheter fluid 132 within the balloon interior 146. Thus, the general description of the energy guides 122A as light guides is not intended to be limiting in any manner, except for as set forth in the claims appended hereto. More particularly, although the catheter systems 100 are often described with the energy source 124 as a light source and the one or more energy guides 122A as light guides, the catheter system 100 can alternatively include any suitable energy source 124 and energy guides 122A for purposes of enabling the generation of the desired plasma in the catheter fluid 132 within the balloon interior 146. For example, in one non-exclusive alternative embodiment, the energy source 124 can be configured to provide high voltage pulses, and each energy guide 122A can include an electrode pair including spaced apart electrodes that extend into the balloon interior 146. In such embodiment, each pulse of high voltage is applied to the electrodes and forms an electrical arc across the electrodes, which, in turn, generates the plasma and forms the pressure waves in the catheter fluid 132 that are utilized to provide the fracture force onto the vascular lesions 106A at the treatment site 106. Still alternatively, the energy source 124 and/or the energy guides 122A can have another suitable design and/or configuration.

In certain embodiments, the energy guides 122A can include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide energy along its length from a guide proximal end 122P to the guide distal end 122D, with the guide distal end 122D having at least one optical window (not shown) that is positioned within the balloon interior 146.

The energy guides 122A can assume many configurations about and/or relative to the catheter shaft 110 of the catheter 102. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A can be disposed within one or more energy guide lumens within the catheter shaft 110.

The energy guides 122A can also be disposed at any suitable positions about the circumference of the guidewire lumen 118 and/or the catheter shaft 110, and the guide distal end 122D of each of the energy guides 122A can be disposed at any suitable longitudinal position relative to the length 142 of the balloon 104 and/or relative to the length of the guidewire lumen 118 (within any suitable or desired emitter station 180) to more effectively and more precisely impart pressure waves for purposes of disrupting the vascular lesions 106A at the treatment site 106.

In certain embodiments, the energy guides 122A can include one or more photoacoustic transducers 153, where each photoacoustic transducer 153 can be in optical communication with the energy guide 122A within which it is disposed. In some embodiments, the photoacoustic transducers 153 can be in optical communication with the guide distal end 122D of the energy guide 122A. In such embodiments, the photoacoustic transducers 153 can have a shape that corresponds with and/or conforms to the guide distal end 122D of the energy guide 122A.

The photoacoustic transducer 153 is configured to convert light energy into an acoustic wave at or near the guide distal end 122D of the energy guide 122A. The direction of the acoustic wave can be tailored by changing an angle of the guide distal end 122D of the energy guide 122A.

In certain embodiments, the photoacoustic transducers 153 disposed at the guide distal end 122D of the energy guide 122A can assume the same shape as the guide distal end 122D of the energy guide 122A. For example, in certain non-exclusive embodiments, the photoacoustic transducer 153 and/or the guide distal end 122D can have a conical shape, a convex shape, a concave shape, a bulbous shape, a square shape, a stepped shape, a half-circle shape, an ovoid shape, and the like. The energy guide 122A can further include additional photoacoustic transducers 153 disposed along one or more side surfaces of the length of the energy guide 122A.

In some embodiments, the energy guides 122A can further include one or more diverting structures or “diverters” (not shown in FIG. 1), such as within the energy guide 122A and/or near the guide distal end 122D of the energy guide 122A, that are configured to direct energy from the energy guide 122A toward a side surface which can be located at or near the guide distal end 122D of the energy guide 122A, before the energy is directed toward the balloon wall 130. A diverting structure can include any structure of the system that diverts energy from the energy guide 122A away from its axial path toward a side surface of the energy guide 122A. The energy guides 122A can each include one or more optical windows disposed along the longitudinal or circumferential surfaces of each energy guide 122A and in optical communication with a diverting structure. Stated in another manner, the diverting structures can have any suitable structural configuration that is configured to direct energy in the energy guide 122A toward a side surface that is at or near the guide distal end 122D, where the side surface is in optical communication with an optical window. The optical windows can include a portion of the energy guide 122A that allows energy to exit the energy guide 122A from within the energy guide 122A, such as a portion of the energy guide 122A lacking a cladding material on or about the energy guide 122A.

Examples of the diverting structures suitable for use include a reflecting element, a refracting element, and a fiber diffuser. The diverting structures suitable for focusing energy away from the tip of the energy guides 122A can include, but are not to be limited to, those having a convex surface, a gradient-index (GRIN) lens, and a mirror focus lens. Upon contact with the diverting structure, the energy is diverted within the energy guide 122A to one or more of the plasma generator 133 and the photoacoustic transducer 153 that is in optical communication with a side surface of the energy guide 122A. When utilized, the plasma generator 133 receives energy emitted from the guide distal end 122D of the energy guide 122A to generate plasma in the catheter fluid 132 within the balloon interior 146, which, in turn, causes the creation of plasma bubbles and/or pressure waves that can be directed away from the side surface of the energy guide 122A and toward the balloon wall 130. Additionally, or in the alternative, when utilized, the photoacoustic transducer 153 converts light energy into an acoustic wave that extends away from the side surface of the energy guide 122A.

The source manifold 136 can be positioned at or near the proximal portion 114 of the catheter system 100. The source manifold 136 can include one or more proximal end openings that can receive the plurality of energy guides 122A of the energy guide bundle 122, the guidewire 112, and/or an inflation conduit 140 that is coupled in fluid communication with the fluid pump 138. The catheter system 100 can also include the fluid pump 138 that is configured to inflate the balloon 104 with the catheter fluid 132 as needed.

As noted above, in the embodiment illustrated in FIG. 1, the system console 123 includes one or more of the energy source 124, the power source 125, the system controller 126, the GUI 127, and the multiplexer 128. Alternatively, the system console 123 can include more components or fewer components than those specifically illustrated in FIG. 1. For example, in certain non-exclusive alternative embodiments, the system console 123 can be designed without the GUI 127. Still alternatively, one or more of the energy source 124, the power source 125, the system controller 126, the GUI 127 and the multiplexer 128 can be provided within the catheter system 100 without the specific need for the system console 123.

As shown, the system console 123, and the components included therewith, is operatively coupled to the catheter 102, the energy guide bundle 122, and the remainder of the catheter system 100. For example, in some embodiments, as illustrated in FIG. 1, the system console 123 can include a console connection aperture 148 (also sometimes referred to generally as a “socket” or a “console receptacle”) by which the energy guide bundle 122 is mechanically coupled to the system console 123. In such embodiments, the energy guide bundle 122 can include an optical connector assembly having a guide coupling housing 150 (also sometimes referred to generally as a “connector housing”) that houses a portion, such as the guide proximal end 122P, of each of the energy guides 122A. At least a portion of the guide coupling housing 150 is configured to fit and be selectively retained within the console connection aperture 148 to provide the mechanical coupling between the energy guide bundle 122 and the system console 123.

The energy guide bundle 122 can also include a guide bundler 152 (or “shell”) that brings each of the individual energy guides 122A closer together so that the energy guides 122A and/or the energy guide bundle 122 can be in a more compact form as it extends with the catheter 102 into the blood vessel 108 during use of the catheter system 100.

The energy source 124 can be selectively and/or alternatively coupled in optical communication with each of the energy guides 122A in the energy guide bundle 122. In particular, the energy source 124 is configured to generate energy in the form of a source beam 124A, such as a pulsed source beam, that can be selectively and/or alternatively directed to and received by each of the energy guides 122A in the energy guide bundle 122. More specifically, as described in greater detail herein below, the source beam 124A from the energy source 124 is directed through the multiplexer 128 such that individual guide beams 124B (or “multiplexed beams”) can be selectively and/or alternatively directed into and received by each of the energy guides 122A in the energy guide bundle 122. In particular, each pulse of the energy source 124 and/or each pulse of the source beam 124A can be directed through the multiplexer 128 to generate a separate guide beam 1248 that is selectively and/or alternatively directed onto one of the energy guides 122A in the energy guide bundle 122. As such, the energy source 124, through use and/or application of the multiplexer 128, can be utilized to energize any of the emitters 135 at any of the emitter stations 180 that may be included within the catheter system 100. Alternatively, the catheter system 100 can include more than one energy source 124. For example, in one non-exclusive alternative embodiment, the catheter system 100 can include a separate energy source 124 for each of the energy guides 122A in the energy guide bundle 122.

The energy source 124 can have any suitable design. In certain embodiments, the energy source 124 can be configured to provide sub-millisecond pulses of energy from the energy source 124 that are focused onto a small spot in order to couple it into the guide proximal end 122P of the energy guide 122A. Such pulses of energy are then directed and/or guided along the energy guides 122A to a location within the balloon interior 146 of the balloon 104, thereby inducing plasma formation in the catheter fluid 132 within the balloon interior 146 of the balloon 104, such as via the plasma generator 133 that can be located at or near the guide distal end 122D of the energy guide 122A. In particular, the energy emitted at the guide distal end 122D of the energy guide 122A is directed toward and impinges on and energizes the plasma generator 133 to form the plasma in the catheter fluid 132 within the balloon interior 146. The plasma formation causes rapid bubble formation, and imparts pressure waves upon the treatment site 106. An exemplary plasma-induced bubble 134 is illustrated in FIG. 1.

As utilized herein, the guide distal end 122D of the energy guide 122A and the corresponding plasma generator 133 can be referred to collectively as an emitter 135. In some applications, one or more emitters 135 that are positioned at approximately the same longitudinal position within the balloon interior 146 relative to the length 142 of the balloon 104 can be referred to as an “emitter station”, such as the one or more emitter stations 180 included as part of the emitter system 131 illustrated in FIG. 1.

In various embodiments, the catheter system 100 is configured to provide a means to power multiple emitter stations 180 in a pressure wave-generating device that is designed to impart pressure onto and induce fractures in vascular lesions 106A, such as calcified vascular lesions and/or fibrous vascular lesions, at the treatment site 106.

In many embodiments, the one or more emitters 135 and/or the emitter stations 180 can be formed from certain materials such that the emitters 135 and/or the emitter stations 180 are more visible to the user or operator during use of the catheter system 100. For example, in many embodiments, the one or more emitters 135 and/or the emitter stations 180 can be formed from and/or include a radiopaque material that is easily visible when used with fluoroscopy during an intravascular lithotripsy procedure. Thus, the visibility of the emitters 135 and/or the emitter stations 180 enables the user or operator to more precisely position the emitters 135 and/or emitter stations 180 as desired substantially adjacent to the vascular lesions 106A, and/or to selectively activate only those emitter stations 180 that are positioned most proximate to the vascular lesions 106A in order to more effectively disrupt the vascular lesions 106A at the treatment site 106. Alternatively, the emitters 135 and/or the emitter stations 180 can be formed from other suitable materials that can be made visible to the user or operator during an intravascular lithotripsy procedure.

In various non-exclusive alternative embodiments, the sub-millisecond pulses of energy from the energy source 124 can be delivered to the treatment site 106 at a frequency of between approximately one hertz (Hz) and 5000 Hz, between approximately Hz and 1000 Hz, between approximately ten Hz and 100 Hz, or between approximately one Hz and 30 Hz. Alternatively, the sub-millisecond pulses of energy can be delivered to the treatment site 106 at a frequency that can be greater than 5000 Hz or less than one Hz, or any other suitable range of frequencies.

It is appreciated that although the energy source 124 is typically utilized to provide pulses of energy, the energy source 124 can still be described as providing a single source beam 124A, such as a single pulsed source beam.

The energy sources 124 suitable for use can include various types of light sources including lasers and lamps. Alternatively, the energy sources 124 can include any suitable type of energy source.

Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (μs) timescales. It is appreciated that there are many combinations of laser wavelengths, pulse widths and energy levels that can be employed to achieve plasma in the catheter fluid 132 of the catheter 102. In various non-exclusive alternative embodiments, the pulse widths can include those falling within a range including from at least ten ns to 3000 ns, at least 20 ns to 100 ns, or at least one ns to 500 ns. Alternatively, any other suitable pulse width range can be used.

Exemplary nanosecond lasers can include those within the UV to IR spectrum, spanning wavelengths of about ten nanometers (nm) to one millimeter (mm). In some embodiments, the energy sources 124 suitable for use in the catheter systems 100 can include those capable of producing light at wavelengths of from at least 750 nm to 2000 nm. In other embodiments, the energy sources 124 can include those capable of producing light at wavelengths of from at least 700 nm to 3000 nm. In still other embodiments, the energy sources 124 can include those capable of producing light at wavelengths of from at least 100 nm to ten micrometers (μm). Nanosecond lasers can include those having repetition rates of up to 200 kHz.

In some embodiments, the laser can include a Q-switched thulium:yttrium-aluminum-garnet (Tm:YAG) laser. In other embodiments, the laser can include a neodymium:yttrium-aluminum-garnet (Nd:YAG) laser, holmium:yttrium-aluminum-garnet (Ho:YAG) laser, erbium:yttrium-aluminum-garnet (Er:YAG) laser, excimer laser, helium-neon laser, carbon dioxide laser, as well as doped, pulsed, fiber lasers.

In still other embodiments, the energy source 124 can include a plurality of lasers that are grouped together in series. In yet other embodiments, the energy source 124 can include one or more low energy lasers that are fed into a high energy amplifier, such as a master oscillator power amplifier (MOPA). In still yet other embodiments, the energy source 124 can include a plurality of lasers that can be combined in parallel or in series to provide the energy needed to create the plasma bubble 134 in the catheter fluid 132.

The catheter system 100 can generate pressure waves having maximum pressures in the range of at least one megapascal (MPa) to 100 MPa. The maximum pressure generated by a particular catheter system 100 will depend on the energy source 124, the absorbing material, the bubble expansion, the propagation medium, the balloon material, and other factors. In various non-exclusive alternative embodiments, the catheter systems 100 can generate pressure waves having maximum pressures in the range of at least approximately two MPa to 50 MPa, at least approximately two MPa to MPa, or at least approximately 15 MPa to 25 MPa.

The pressure waves can be imparted upon the treatment site 106 from a distance within a range from at least approximately 0.1 millimeters (mm) to greater than approximately 25 mm extending radially from the energy guides 122A when the catheter 102 is placed at the treatment site 106. In various non-exclusive alternative embodiments, the pressure waves can be imparted upon the treatment site 106 from a distance within a range from at least approximately ten mm to 20 mm, at least approximately one mm to ten mm, at least approximately 1.5 mm to four mm, or at least approximately 0.1 mm to ten mm extending radially from the energy guides 122A when the catheter 102 is placed at the treatment site 106. In other embodiments, the pressure waves can be imparted upon the treatment site 106 from another suitable distance that is different than the foregoing ranges. In some embodiments, the pressure waves can be imparted upon the treatment site 106 within a range of at least approximately two MPa to MPa at a distance from at least approximately 0.1 mm to ten mm. In some embodiments, the pressure waves can be imparted upon the treatment site 106 from a range of at least approximately two MPa to 25 MPa at a distance from at least approximately 0.1 mm to ten mm. Still alternatively, other suitable pressure ranges and distances can be used.

The power source 125 is electrically coupled to and is configured to provide necessary power to each of the energy source 124, the system controller 126, the GUI 127, the multiplexer 128, and the handle assembly 129. The power source 125 can have any suitable design for such purposes.

The system controller 126 is electrically coupled to and receives power from the power source 125. The system controller 126 is coupled to and is configured to control operation of each of the energy source 124, the GUI 127 and the multiplexer 128. The system controller 126 can include one or more processors or circuits for purposes of controlling the operation of at least the energy source 124, the GUI 127 and the multiplexer 128. For example, the system controller 126 can control the energy source 124 for generating pulses of energy as desired and/or at any desired firing rate. Subsequently, the system controller 126 can then control the multiplexer 128 so that the energy from the energy source 124, as the source beam 124A, can be selectively and/or alternatively directed to each of the energy guides 122A, such as in the form of individual guide beams 124B, in any desired firing sequence or firing pattern.

More specifically, the system controller 126 can control the energy source 124 and/or the multiplexer 128 so that individual guide beams 124B can be directed to each of the energy guides 122A, or sets or subsets of the energy guides 122A, in any desired firing sequence, firing pattern, firing order, firing energy levels (which can be influenced by any or all of pulse width, pulse amplitude and/or pulse wavelength) and/or firing rate. As such, the system controller 126 can control the energy source 124 and/or the multiplexer 128 so that individual guide beams 124B can be directed to any of the emitter stations 180 and/or any of the emitters 135 incorporated within any of the emitter stations 180 in any desired firing sequence, firing pattern, firing order, firing energy levels and/or firing rate. For example, in a catheter system 100 that includes eight energy guides 122A that are arranged in a linear pattern with angular orientation spiraling around the guidewire lumen 118, the system controller 126 can control the sequencing of the firing of the energy from the energy source 124 to each of the energy guides 122A, or sets or subsets thereof, in any desired manner. As used herein, the term “firing rate” is intended to mean the number of pulses per a given time frame. Further, as used herein, the term “firing energy level” is intended to mean the intensity of the energy pulse, which can be varied depending upon the pulse width and/or the pulse amplitude of any or all of the energy pulse(s). Certain non-exclusive examples of alternative applications of sequencing of the firing of the energy guides 122A and/or the emitters 135 within a given emitter station 180 will be described in detail herein below.

The system controller 126 can further be configured to control operation of other components of the catheter system 100, such as the positioning of the catheter 102, the guide distal end 122D of the energy guides 122A, and/or the emitters 135 (or emitter stations 180) adjacent to the treatment site 106, the inflation of the balloon 104 with the catheter fluid 132, etc. Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 129.

The GUI 127 is accessible by the user or operator of the catheter system 100. The GUI 127 is electrically connected to the system controller 126. With such design, the GUI 127 can be used by the user or operator to ensure that the catheter system 100 is effectively utilized to impart pressure onto and induce fractures into the vascular lesions 106A at the treatment site 106. More particularly, in certain embodiments, the GUI 127 can enable the user or operator to select and/or deselect any of the emitter stations 180 and individual emitters 135 in order to more effectively and efficiently generate plasma in the catheter fluid 132 within the balloon interior 146, and, thus, impart pressure onto and induce fractures into the vascular lesions 106A at the treatment site 106.

The GUI 127 can provide the user or operator with information that can be used before, during and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time during use of the catheter system 100. In various embodiments, the GUI 127 can include one or more colors, different sizes, varying brightness, etc., that may act as alerts to the user or operator. Additionally, or in the alternative, the GUI 127 can provide audio data or information to the user or operator. The specifics of the GUI 127 can vary depending upon the design requirements of the catheter system 100, or the specific needs, specifications and/or desires of the user or operator.

The multiplexer 128 is configured to selectively and/or alternatively direct energy from the energy source 124 to each of the energy guides 122A in the energy guide bundle 122. More particularly, the multiplexer 128 is configured to receive energy from the energy source 124, such as in the form of a single source beam 124A from a single laser source, and selectively and/or alternatively direct such energy in the form of individual guide beams 124B, as desired, to each of the energy guides 122A in the energy guide bundle 122. As such, the multiplexer 128 enables a single energy source 124 to be channeled separately in any desired sequence or pattern through a plurality of energy guides 122A such that the catheter system 100 is able to impart pressure onto and induce fractures in vascular lesions 106A at the treatment site 106 within or adjacent to a vessel wall 108A of the blood vessel 108 in a desired manner. As shown, in certain embodiments, the catheter system 100 can include one or more optical elements 147 for purposes of directing the energy, such as the source beam 124A, from the energy source 124 to the multiplexer 128.

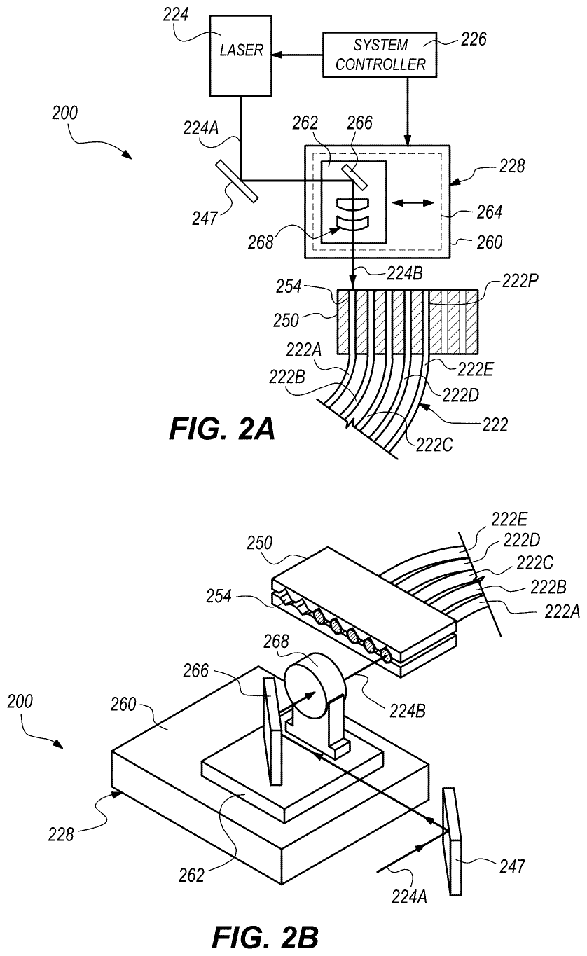

The multiplexer 128 can have any suitable design for purposes of selectively and/or alternatively directing the energy from the energy source 124 to each of the energy guides 122A of the energy guide bundle 122. Various non-exclusive alternative embodiments of the multiplexer 128 are described in detail herein below in relation to FIGS. 2A-7.

As shown in FIG. 1, the handle assembly 129 can be positioned at or near the proximal portion 114 of the catheter system 100. In this embodiment, the handle assembly 129 is coupled to the balloon 104 and is positioned spaced apart from the balloon 104. Alternatively, the handle assembly 129 can be positioned at another suitable location.

The handle assembly 129 is attached to the catheter shaft 110 and is handled and used by the user or operator to operate, position and control the catheter 102. The design and specific features of the handle assembly 129 can vary to suit the design requirements of the catheter system 100. In the embodiment illustrated in FIG. 1, the handle assembly 129 is separate from, but in electrical and/or fluid communication with one or more of the system controller 126, the energy source 124, the fluid pump 138, and the GUI 127.

In some embodiments, the handle assembly 129 can integrate and/or include at least a portion of the system controller 126 within an interior of the handle assembly 129. For example, as shown, in certain embodiments, the handle assembly 129 can include circuitry 155, which is electrically coupled between catheter electronics and the system console 123, and which can form at least a portion of the system controller 126. In one embodiment, the circuitry 155 can include a printed circuit board having one or more integrated circuits, or any other suitable circuitry. In an alternative embodiment, the circuitry 155 can be omitted, or can be included within the system controller 126, which in various embodiments can be positioned outside of the handle assembly 129, such as within the system console 123. It is understood that the handle assembly 129 can include fewer or additional components than those specifically illustrated and described herein.

The emitter system 131 includes one or more emitter stations 180 (and preferably a plurality of emitter stations 180), with each emitter station 180 including one or more emitters 135 (and preferably a plurality of emitters 135). As noted, each of the emitters 135 includes the guide distal end 122D of one of the energy guides 122A, and the corresponding plasma generator 133. As referred to herein, the “plasma generator” can include and/or incorporate any suitable type of structure that is located at or near the guide distal end 122D of the energy guide 122A. In many embodiments, the plasma generator 133 can be positioned slightly spaced apart from the guide distal end 122D of the energy guide 122A. In certain embodiments, the plasma generator 133 can be provided in the form of a backstop-type structure with an angled face that redirects energy emitted from the guide distal end 122D toward the balloon wall 130 of the balloon 104 and/or toward the vessel wall 108A of the blood vessel 108 at the treatment site 106.

Each of the emitters 135 is configured to selectively receive energy from the energy source 124, under control of the system controller 126 and as directed by the multiplexer 128, and emit the energy from the guide distal end 122D toward the plasma generator 133. The energy emitted from the guide distal end 122D impinges upon and energizes material of the plasma generator 133, such as material on the angled face of the plasma generator 133, for purposes of generating plasma in the catheter fluid 132 within the balloon interior 146. The plasma generation ionizes and/or superheats the surrounding catheter fluid 132 and thus causes rapid inertial bubble formation, and imparts pressure waves upon the treatment site 106.

The plasma generator 133 can be formed from any suitable materials. For example, in certain non-exclusive embodiments, the plasma generator 133 can be formed from one or more metals such as titanium, stainless steel, tungsten, tantalum, platinum, molybdenum, niobium, iridium, etc. Alternatively, the plasma generator 133 may be formed from plastics such as polyimide and nylon. Still alternatively, the plasma generator 133 can be formed from other suitable materials.

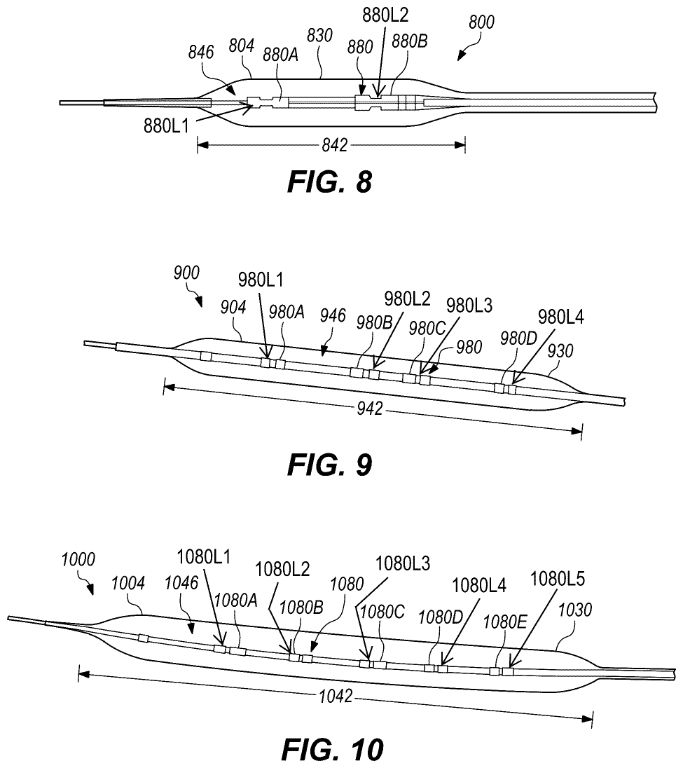

Further details of various embodiments of the emitter system 131, the emitter stations 180 and/or the individual emitters 135 will be provided herein below in relation to FIGS. 8-10.

The catheter system 100 can also include the fluid pump 138 that is configured to inflate the balloon 104 with the catheter fluid 132 as needed.

As with all embodiments illustrated and described herein, various structures may be omitted from the figures for clarity and ease of understanding. Further, the figures may include certain structures that can be omitted without deviating from the intent and scope of the invention.

FIG. 2A is a simplified schematic top view illustration of a portion of an embodiment of the catheter system 200. More particularly, FIG. 2A illustrates a plurality of energy guides, such as a first energy guide 222A, a second energy guide 222B, a third energy guide 222C, a fourth energy guide 222D and a fifth energy guide 222E, an energy source 224, a system controller 226, and an embodiment of the multiplexer 228 that receives energy in the form of a source beam 224A, such as a pulsed source beam, from the energy source 224 and selectively and/or alternatively directs the energy in the form of individual guide beams 224B in any desired sequence and/or pattern to any or all of the energy guides 222A-222E under control of the system controller 226. The energy guides 222A-222E, the energy source 224 and the system controller 226 are substantially similar in design and function as described in detail herein above. Accordingly, such components will not be described in detail in relation to the embodiment illustrated in FIG. 2A. It is further appreciated that certain components of the system console 123 illustrated and described above in relation to FIG. 1, such as the power source 125 and the GUI 127, are not illustrated in FIG. 2A for purposes of simplicity and ease of illustration, but would typically be included in many embodiments.

As noted above, the multiplexer 228 is configured to receive energy in the form of the source beam 224A from the energy source 224 and selectively and/or alternatively direct the energy in the form of individual guide beams 224B in any desired sequence and/or pattern to any or all of the energy guides 222A-222E. As such, as shown in FIG. 2A, the multiplexer 228 is operatively and/or optically coupled in optical communication to the energy guide bundle 222 and/or to each of the plurality of energy guides 222A-222E.

As illustrated, a guide proximal end 222P of each of the plurality of energy guides 222A-222E is retained within a guide coupling housing 250, such as within guide coupling slots 254 that are formed into the guide coupling housing 250. In various embodiments, the guide coupling housing 250 is configured to be selectively coupled to the system console 123 (illustrated in FIG. 1) so that the guide coupling slots 254, and thus the energy guides 222A-222E, are maintained in a desired fixed position relative to the multiplexer 228 and/or the system console 123 during use of the catheter system 200. In some embodiments, the guide coupling slots 254 are provided in the form of V-grooves, such as in a V-groove ferrule block commonly used in multichannel fiber optics communication systems. Alternatively, the guide coupling slots 254 can have another suitable design.

It is appreciated that the guide coupling housing 250 can have any suitable number of guide coupling slots 254, which can be positioned and/or oriented relative to one another in any suitable manner to best align the guide coupling slots 254 and thus the energy guides 222A-222E relative to the multiplexer 228. In the embodiment illustrated in FIG. 2A, the guide coupling housing 250 includes seven guide coupling slots 254 that are spaced apart in a linear arrangement relative to one another, with precise interval spacing between adjacent guide coupling slots 254. Thus, in such embodiment, the guide coupling housing 250 is capable of retaining the guide proximal end 222P of up to seven energy guides (although only five energy guides 222A-222E are specifically shown in FIG. 2A). Alternatively, the guide coupling housing 250 can have a different number of guide coupling slots, greater than seven or less than seven, and/or the guide coupling slots 254 can be arranged in a different manner relative to one another.

The design of the multiplexer 228 can be varied depending on the requirements of the catheter system 200, the relative positioning of the energy guides 222A-222E, and/or to suit the desires of the user or operator of the catheter system 200. In the embodiment illustrated in FIG. 2A, the multiplexer 228 includes one or more of a multiplexer base 260, a multiplexer stage 262, a stage mover 264 (illustrated in phantom), a redirector 266, and coupling optics 268. Alternatively, the multiplexer 228 can include more components or fewer components than those specifically illustrated in FIG. 2A.

During use of the catheter system 200, the multiplexer base 260 is fixed in position relative to the energy source 224 and the energy guides 222A-222E. In this embodiment, the multiplexer stage 262 is movably supported on the multiplexer base 260. More particularly, the stage mover 264 is configured to move the multiplexer stage 262 relative to the multiplexer base 260. As shown in FIG. 2A, the redirector 266 and the coupling optics 268 are mounted on and/or retained by the multiplexer stage 262. Thus, movement of the multiplexer stage 262 relative to the multiplexer base 260 results in corresponding movement of the redirector 266 and the coupling optics 268 relative to the fixed multiplexer base 260. With the energy guides 222A-222E being fixed in position relative to the multiplexer base 260, movement of the multiplexer stage 262 results in corresponding movement of the redirector 266 and the coupling optics 268 relative to the energy guides 222A-222E.

In various embodiments, the multiplexer 228 is configured to precisely align the coupling optics 268 with each of the energy guides 222A-222E such that the source beam 224A generated by the energy source 224 can be precisely directed and focused by the multiplexer 228 as a corresponding guide beam 224B to each of the energy guides 222A-222E. In its simplest form, as shown in FIG. 2A, the multiplexer 228 uses a precision mechanism, such as the stage mover 264, to translate the coupling optics 268 along a linear path. This approach requires a single degree of freedom. In certain embodiments, the linear translation mechanism, such as the stage mover 264, and/or the multiplexer stage 262 can be equipped with mechanical stops so that the coupling optics 268 can be precisely aligned with the position of each of the energy guides 222A-222E in any desired sequence and/or pattern. Alternatively, the stage mover 264 can be electronically controlled to line the beam path of the guide beam 224B in any desired sequence and/or pattern with each individual energy guide 222A-222E that is retained, in part, within the guide coupling housing 250.

As noted above, the multiplexer stage 262 is configured to carry the necessary optics, such as the redirector 266 and the coupling optics 268, to direct and focus the energy generated by the energy source 224 onto each energy guide 222A-222E for optimal coupling. With such design, the low divergence of the guide beam 224A over the short distance of motion of the translated multiplexer stage 262 has minimum impact on coupling efficiency to the energy guide 222A-222E.

During operation, the stage mover 264 drives the multiplexer stage 262 to align the beam path of the guide beam 224B with a selected energy guide 222A-222E and then the system controller 226 fires the energy source 224 in pulsed or semi-CW mode. The stage mover 264 then steps the multiplexer stage 262 to the next stop, i.e. to the next desired energy guide 222A-222E, and the system controller 226 again fires the energy source 224. This process is repeated as desired so that energy in the form of the guide beams 224B is directed onto any or all of the energy guides 222A-222E in a desired sequence and/or pattern. It is appreciated that the stage mover 264 can move the multiplexer stage 262 so that it is aligned with any of the energy guides 222A-222E, then the system controller 226 fires the energy source 224. In this manner, the multiplexer 228 can achieve sequence firing through the energy guides 222A-222E or fire in any desired pattern relative to the energy guides 222A-222E.

In this embodiment, the stage mover 264 can have any suitable design for purposes of moving the multiplexer stage 262 in a linear manner relative to the multiplexer base 260. More particularly, the stage mover 264 can be any suitable type of linear translation mechanism.

As shown in FIG. 2A, the catheter system 200 can further include an optical element 247, such as a reflecting or redirecting element such as a mirror, that reflects the source beam 224A from the energy source 224 so that the source beam 224A is directed toward the multiplexer 228. In one embodiment, as shown, the optical element 247 can be positioned along the beam path to redirect the source beam 224A by approximately degrees so that the source beam 224A is directed toward the multiplexer 228. Alternatively, the optical elements 247 can redirect the source beam 224A by more than degrees or less than 90 degrees. Still alternatively, the catheter system 200 can be designed without the optical elements 247, and the energy source 224 can direct the source beam 224A directly toward the multiplexer 228.

In this embodiment, the source beam 224A being directed toward the multiplexer 228 initially impinges on the redirector 266, which is configured to redirect the source beam 224A toward the coupling optics 268. In some embodiments, the redirector 266 redirects the source beam 224A by approximately 90 degrees toward the coupling optics 268. Alternatively, the redirector 266 can redirect the source beam 224A by more than degrees or less than 90 degrees toward the coupling optics 268. Thus, the redirector 266 that is mounted on the multiplexer stage 262 is configured to direct the source beam 224A through the coupling optics 268 so that individual guide beams 224B are focused into the individual energy guides 222A-222E in the guide coupling housing 250.

The coupling optics 268 can have any suitable design for purposes of focusing the individual guide beams 224B onto each of the energy guides 222A-222E. In one embodiment, the coupling optics 268 include two lenses that are specifically configured to focus the individual guide beams 224B as desired. Alternatively, the coupling optics 268 can have another suitable design.

In certain non-exclusive alternative embodiments, the steering of the source beam 224A so that it is properly directed and focused onto each of the energy guides 222A-222E can be accomplished using mirrors that are attached to optomechanical scanners, X-Y galvanometers or other multi-axis beam steering devices.

Still alternatively, although FIG. 2A illustrates that the energy guides 222A-222E are fixed in position relative to the multiplexer base 260, in some embodiments, the energy guides 222A-222E can be configured to move relative to coupling optics 268 that are fixed in position. In such embodiments, the guide coupling housing 250 itself would move, such as the guide coupling housing 250 can be carried by a linear translation stage, and the system controller 226 can control the linear translation stage to move in a stepped manner so that the energy guides 222A-222E are each aligned, in a desired pattern, with the coupling optics and the guide beams 224B. While such an embodiment can be effective, it is further appreciated that additional protection and controls would be required to make it safe and reliable as the guide coupling housing 250 moves relative to the coupling optics 268 of the multiplexer 228 during use.

FIG. 2B is a simplified schematic perspective view illustration of a portion of the catheter system 200 and the multiplexer 228 illustrated in FIG. 2A. In particular, FIG. 2B illustrates another view of the guide coupling housing 250, with the guide coupling slots 254, that is configured to retain a portion of each of the energy guides 222A-222E; the optical element 247 that initially redirects the source beam 224A from the energy source 224 (illustrated in FIG. 2A) toward the multiplexer 228; and the multiplexer 228, including the multiplexer base 260, the multiplexer stage 262, the redirector 266 and the coupling optics 268, that receives the source beam 224A and then directs and focuses individual guide beams 224B in any desired sequence and/or pattern toward any or all of the energy guides 222A-222E. It is appreciated that the stage mover 264 is not illustrated in FIG. 2B for purposes of simplicity and ease of illustration.

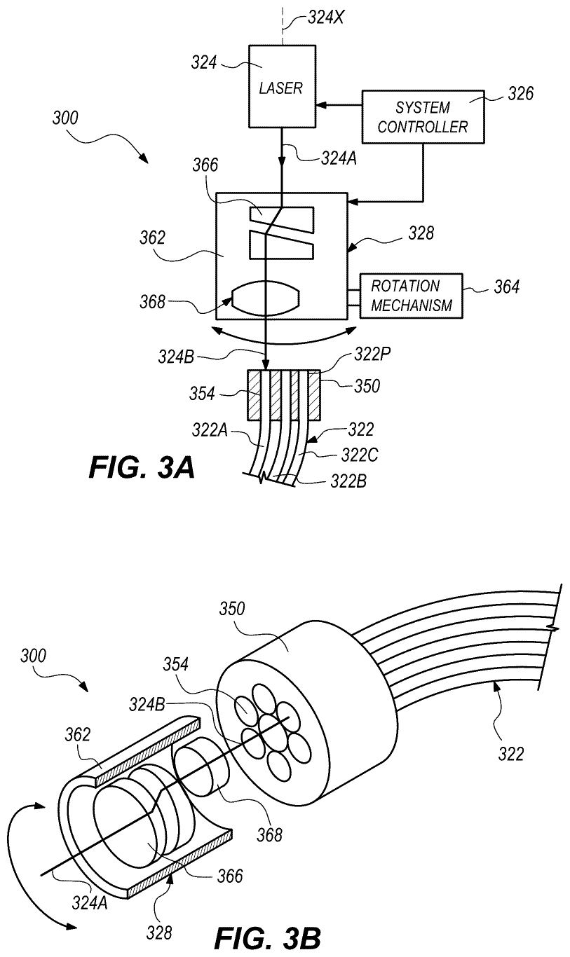

FIG. 3A is a simplified schematic top view illustration of a portion of an embodiment of the catheter system 300 including another embodiment of the multiplexer 328. More particularly, FIG. 3A illustrates a plurality of energy guides, such as a first energy guide 322A, a second energy guide 322B and a third energy guide 322C, an energy source 324, a system controller 326, and the multiplexer 328 that receives energy in the form of a source beam 324A from the energy source 324 and selectively and/or alternatively directs the energy in the form of individual guide beams 324B in any desired sequence and/or pattern to each of the energy guides 322A-322C under control of the system controller 326. The energy guides 322A-322C, the energy source 324 and the system controller 326 are substantially similar in design and function as described in detail herein above. Accordingly, such components will not be described in detail in relation to the embodiment illustrated in FIG. 3A. It is further appreciated that certain components of the system console 123 illustrated and described above in relation to FIG. 1, such as the power source 125 and the GUI 127, are not illustrated in FIG. 3A for purposes of simplicity and ease of illustration, but would typically be included in many embodiments.

As with previous embodiments, the multiplexer 328 is configured to receive energy in the form of the source beam 324A, such as a single pulsed source beam, from the energy source 324 and selectively and/or alternatively direct the energy in the form of individual guide beams 324B in any desired sequence and/or pattern to any or all of the energy guides 322A-322C. As such, as shown in FIG. 3A, the multiplexer 328 is operatively and/or optically coupled in optical communication to the energy guide bundle 322 and/or to the plurality of energy guides 322A-322C.

As illustrated, a guide proximal end 322P of each of the plurality of energy guides 322A-322C is retained within a guide coupling housing 350, such as within guide coupling slots 354 that are formed into the guide coupling housing 350. In various embodiments, the guide coupling housing 350 is configured to be selectively coupled to the system console 123 (illustrated in FIG. 1) so that the guide coupling slots 354, and thus the energy guides 322A-322C, are maintained in a desired fixed position relative to the multiplexer 328 and/or the system console 123 during use of the catheter system 300.

Referring now to FIG. 3B, FIG. 3B is a simplified schematic perspective view illustration of a portion of the catheter system 300 and the multiplexer 328 illustrated in FIG. 3A. As shown in FIG. 3B, the guide coupling housing 350 can be substantially cylindrical-shaped. It is appreciated that the guide coupling housing 350 can have any suitable number of guide coupling slots 354, which can be positioned and/or oriented relative to one another in any suitable manner, so as to best align the guide coupling slots 354 and thus the energy guides 322A-322C of the energy guide bundle 322 relative to the multiplexer 328. In the embodiment illustrated in FIG. 3B, the guide coupling housing 350 includes seven guide coupling slots 354 that are arranged in a circular and/or hexagonal packed pattern. Thus, in such embodiment, the guide coupling housing 350 is capable of retaining the guide proximal end of up to seven energy guides. Alternatively, the guide coupling housing 350 can have a different number of guide coupling slots, greater than seven or less than seven, and/or the guide coupling slots 354 can be arranged in a different manner relative to one another, such as in another suitable circular periodic pattern.

Returning to FIG. 3A, in this embodiment, the multiplexer 328 includes one or more of a multiplexer stage 362, a stage mover 364, a redirector 366, and coupling optics 368. Alternatively, the multiplexer 328 can include more components or fewer components than those specifically illustrated in FIG. 3A.

As shown in the embodiment illustrated in FIG. 3A, the stage mover 364 is configured to move the multiplexer stage 362 in a rotational manner. More particularly, in this embodiment, the multiplexer stage 362 and/or the stage mover 364 requires a single rotational degree of freedom. As shown, the multiplexer stage 362 and the guide coupling housing 350 are aligned on a central axis 324X of the energy source 324. As such, the multiplexer stage 362 is configured to be rotated by the stage mover 364 about the central axis 324X.

The redirector 366 and the coupling optics 368 are mounted on and/or retained by the multiplexer stage 362. During use of the catheter system 300, the source beam 324A is initially directed toward the multiplexer 328 and/or the multiplexer stage 362 along the central axis 324X of the energy source 324. Subsequently, the redirector 366 is configured to deviate the source beam 324A a fixed distance laterally, off the central axis 324X of the energy source 324, such that the source beam 324A is directed in a direction that is substantially parallel to and spaced apart from the central axis 324X. More specifically, the redirector 366 deviates the source beam 324A to coincide with the radius of the circular pattern of the energy guides 322A-322C in the guide coupling housing 350. As the multiplexer stage 362 is rotated, the source beam 324A that is directed through the redirector 366 traces out a circular path.

It is appreciated that the redirector 366 can have any suitable design. For example, in certain non-exclusive alternative embodiments, the redirector 366 can be provided in the form of an anamorphic prism pair, a pair of wedge prisms, or a pair of close-spaced right-angle mirrors or prisms. Alternatively, the redirector 366 can include another suitable configuration of optics in order to achieve the desired lateral beam offset.

As noted, the coupling optics 368 are also mounted on and/or retained by the multiplexer stage 362. As with the previous embodiments, the coupling optics 368 are configured to focus the individual guide beams 324B onto each of the energy guides 322A-322C in the energy guide bundle 322 retained, in part, within the guide coupling housing 350 for optimal coupling.

As noted above, the multiplexer 328 is configured to precisely align the coupling optics 368 with each of the energy guides 322A-322C such that the source beam 324A generated by the energy source 324 can be precisely directed and focused by the multiplexer 328 as a corresponding guide beam 324B to each of the energy guides 322A-322C. In certain embodiments, the stage mover 364 and/or the multiplexer stage 362 can be equipped with mechanical stops so that the coupling optics 368 can be precisely aligned with the position of each of the energy guides 322A-322C in any desired sequence and/or pattern. Alternatively, the stage mover 364 can be electronically controlled, such as by using stepper motors or a piezo-actuated rotational stage, to line the beam path of the guide beam 324B in any desired sequence and/or pattern with each individual energy guide 322A-322C that is retained, in part, within the guide coupling housing 350.

During use of the catheter system 300, the stage mover 364 drives the multiplexer stage 362 to couple the guide beam 324B with a selected energy guide 322A-322C and then the system controller 326 fires the energy source 324 in pulsed or semi-CW mode. The stage mover 364 then steps the multiplexer stage 362 angularly to the next stop, i.e. to the next desired energy guide 322A-322C, and the system controller 326 again fires the energy source 324. This process is repeated as desired so that energy in the form of the guide beams 324B is directed onto any or all of the energy guides 322A-322C in a desired sequence and/or pattern. It is appreciated that the stage mover 364 can move the multiplexer stage 362 so that it is aligned with any of the energy guides 322A-322C, then the system controller 326 fires the energy source 324. In this manner, the multiplexer 328 can achieve sequence firing through the energy guides 322A-322C or fire in any desired pattern relative to the energy guides 322A-322C.

In this embodiment, the stage mover 364 can have any suitable design for purposes of moving the multiplexer stage 362 in a rotational manner about the central axis 324X. More particularly, the stage mover 364 can be any suitable type of rotational mechanism.

Alternatively, although FIG. 3A illustrates that the energy guides 322A-322C are fixed in position relative to the multiplexer stage 362, in some embodiments, it is appreciated that the energy guides 322A-322C can be configured to move and/or rotate relative to coupling optics 368 that are fixed in position. In such embodiments, the guide coupling housing 350 itself would move, such as the guide coupling housing 350 can be rotated about the central axis 324X, and the system controller 326 can control the rotational stage to move in a stepped manner so that the energy guides 322A-322C are each aligned, in a desired sequence and/or pattern, with the coupling optics and the guide beams 324B. In such embodiment, the guide coupling housing 350 would not be continuously rotated, but would be rotated a fixed number of degrees and then counter-rotated to avoid the winding of the energy guides 322A-322C.

Returning again to FIG. 3B, FIG. 3B illustrates another view of the guide coupling housing 350, with the guide coupling slots 354, that is configured to retain a portion of each of the energy guides; and the multiplexer 328, including the multiplexer stage 362, the redirector 366 and the coupling optics 368, that receives the source beam 324A and then directs and focuses individual guide beams 324B in any desired sequence and/or pattern toward each of the energy guides. It is appreciated that the stage mover 364 is not illustrated in FIG. 3B for purposes of simplicity and ease of illustration.

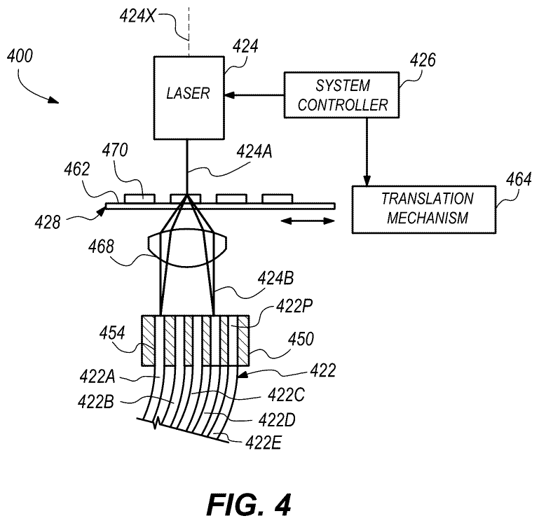

FIG. 4 is a simplified schematic top view illustration of a portion of the catheter system 400 and still another embodiment of the multiplexer 428. More particularly, FIG. 4 illustrates a plurality of energy guides, such as a first energy guide 422A, a second energy guide 422B, a third energy guide 422C, a fourth energy guide 422D and a fifth energy guide 422E, an energy source 424, a system controller 426, and the multiplexer 428 that receives energy in the form of a source beam 424A from the energy source 424 and selectively and/or alternatively directs the energy in the form of individual guide beams 424B in any desired sequence and/or pattern to any or all of the energy guides 422A-422E under control of the system controller 426. The energy guides 422A-422E, the energy source 424 and the system controller 426 are substantially similar in design and function as described in detail herein above. Accordingly, such components will not be described in detail in relation to the embodiment illustrated in FIG. 4. It is further appreciated that certain components of the system console 123 illustrated and described above in relation to FIG. 1, such as the power source 125 and the GUI 127, are not illustrated in FIG. 4 for purposes of simplicity and ease of illustration, but would typically be included in many embodiments.

As noted above, the multiplexer 428 is configured to receive energy in the form of the source beam 424A, such as a single pulsed source beam, from the energy source 424 and selectively and/or alternatively direct the energy in the form of individual guide beams 424B in any desired sequence and/or pattern to any or all of the energy guides 422A-422E. As such, as shown in FIG. 4, the multiplexer 428 is operatively and/or optically coupled in optical communication to the energy guide bundle 422 and/or to the plurality of energy guides 422A-422E.

As illustrated, a guide proximal end 422P of each of the plurality of energy guides 422A-422E is retained within a guide coupling housing 450, such as within guide coupling slots 454 that are formed into the guide coupling housing 450. In various embodiments, the guide coupling housing 450 is configured to be selectively coupled to the system console 123 (illustrated in FIG. 1) so that the guide coupling slots 454, and thus the energy guides 422A-422E, are maintained in a desired fixed position relative to the multiplexer 428 and/or the system console 123 during use of the catheter system 400. It is appreciated that the guide coupling housing 450 can have any suitable number of guide coupling slots 454. In the embodiment illustrated in FIG. 4, five guide coupling slots 454 are visible within the guide coupling housing 450. Thus, in such embodiment, the guide coupling housing 450 is capable of retaining the guide proximal end 422P of up to five energy guides. Alternatively, the guide coupling housing 450 can have a different number of guide coupling slots 454, greater than five or less than five guide coupling slots 454.

In the embodiment illustrated in FIG. 4, the multiplexer 428 includes one or more of a multiplexer stage 462, a stage mover 464, one or more diffractive optical elements 470 (or “DOE”), and coupling optics 468. Alternatively, the multiplexer 428 can include more components or fewer components than those specifically illustrated in FIG. 4.

As shown, the diffractive optical elements 470 are mounted on and/or retained by the multiplexer stage 462. The stage mover 464 is configured to move the multiplexer stage 462, such as translationally, such that each of the one or more diffractive optical elements 470 are selectively and/or alternatively positioned in the beam path of the source beam 424A from the energy source 424.

During use of the catheter system 400, each of the one or more diffractive optical elements 470 is configured to separate the source beam 424A into one, two, three or more individual guide beams 424B. It is appreciated that the diffractive optical elements 470 can have any suitable design. For example, in certain non-exclusive embodiments, the diffractive optical elements 470 can be created using arrays of micro-prisms, micro-lenses, or other patterned diffractive elements.

It is appreciated that there are many possible patterns to organize the energy guides 422A-422E in the guide coupling housing 450 using this approach. The simplest pattern for the energy guides 422A-422E within the guide coupling housing 450 would be a hexagonal, close-packed pattern, similar to what was illustrated in FIGS. 3A and 3B. Alternatively, the energy guides 422A-422E within the guide coupling housing 450 could also be arranged in a square, linear, circular, or other suitable pattern.

As shown in FIG. 4, the guide coupling housing 450 can be aligned on the central axis 424X of the energy source 424, with the diffractive optical elements 470 mounted on the multiplexer stage 462 being inserted along the beam path between the energy source 424 and the guide coupling housing 450. As illustrated, the coupling optics 468 are also positioned along the central axis 424X of the energy source 424, and the coupling optics are positioned between the diffractive optical elements 470 and the guide coupling housing 450.

During operation, the source beam 424A impinging on one of the plurality of diffractive optical elements 470 splits the source beam 424A into two or more deviated beams, i.e. two or more guide beams 424B. These guide beams 424B are, in turn, directed and focused by the coupling optics 468 down onto the individual energy guides 422A-422E that are retained in the guide coupling housing 450. In one configuration, the diffractive optical element 470 would split the source beam 424A into as many energy guides as are present within the single-use device. In such configuration, the power in each guide beam 424B is based on the number of guide beams 424B that are generated from the single source beam 424A minus scattering and absorption losses. Alternatively, the diffractive optical element 470 can be configured to split the source beam 424A so that guide beams 424B are directed into any single energy guide or any selected multiple energy guides. Thus, the multiplexer stage 462 can be configured to retain a plurality of diffractive optical elements 470, such as with multiple diffractive optical element patterns etched on a single plate, to provide options for the user or operator for coupling the guide beams 424B to the desired number and pattern of energy guides. In such embodiments, pattern selection can be achieved by moving the multiplexer stage 462 with the stage mover 464, such as translationally, so that the desired diffractive optical element 470 is positioned in the beam path of the source beam 424A between the energy source 424 and the coupling optics 468.

As with the previous embodiments, the coupling optics 468 can have any suitable design for purposes of focusing the individual guide beams 424B, or multiple guide beams 424B simultaneously, onto the desired energy guides 422A-422E.

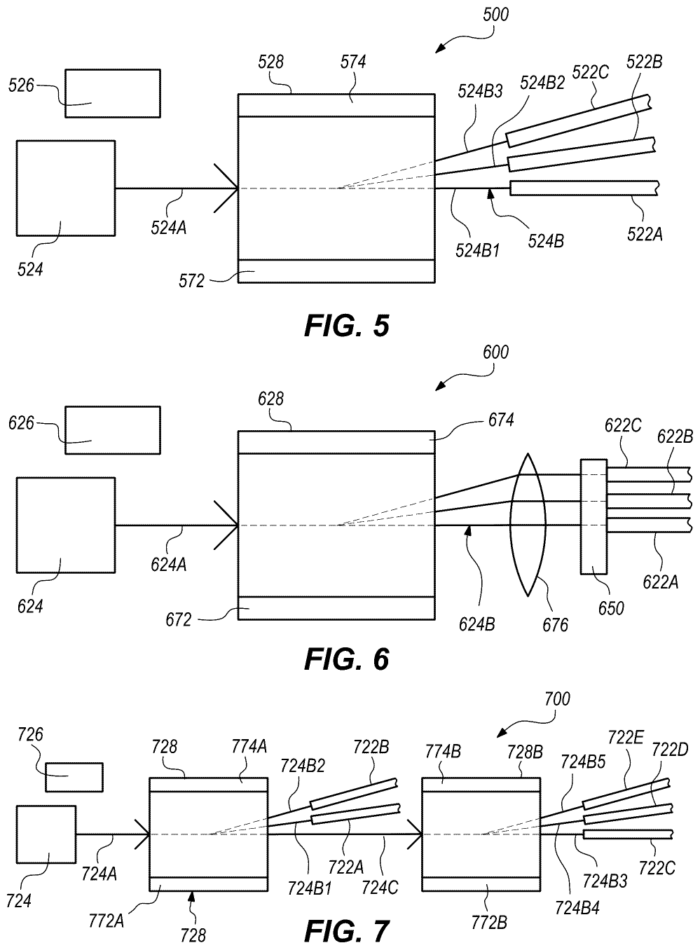

FIG. 5 is a simplified schematic top view illustration of a portion of the catheter system 500 and yet another embodiment of the multiplexer 528. More particularly, Figure illustrates a plurality of energy guides, such as a first energy guide 522A, a second energy guide 522B and a third energy guide 522C, an energy source 524, a system controller 526, and the multiplexer 528 that receives energy in the form of a source beam 524A from the energy source 524 and selectively and/or alternatively directs the energy in the form of individual guide beams 524B in any desired sequence and/or pattern to any or all of the energy guides 522A-522C under control of the system controller 526. The energy guides 522A-522C, the energy source 524 and the system controller 526 are substantially similar in design and function as described in detail herein above. Accordingly, such components will not be described in detail in relation to the embodiment illustrated in FIG. 5. It is further appreciated that certain components of the system console 123 illustrated and described above in relation to FIG. 1, such as the power source 125 and the GUI 127, are not illustrated in FIG. 5 for purposes of simplicity and ease of illustration, but would typically be included in many embodiments.

As noted above, the multiplexer 528 is configured to receive energy in the form of the source beam 524A, such as a single pulsed source beam, from the energy source 524 and selectively and/or alternatively direct the energy in the form of individual guide beams 524B in any desired sequence and/or pattern to any or all of the energy guides 522A-522C. As such, as shown in FIG. 5, the multiplexer 528 is operatively and/or optically coupled in optical communication to the plurality of energy guides 522A-522C.

However, as illustrated in FIG. 5, the multiplexer 528 has a different design than any of the previous embodiments. In some embodiments, it may be desirable to design the multiplexer 528 to receive the source beam 524A from a single energy source 524 and selectively and/or alternatively direct the energy in the form of individual guide beams 524B in any desired sequence and/or pattern to any or all of the energy guides 522A-522C in a manner that is easily reconfigurable and that does not involve moving parts. For example, using an acousto-optic deflector (AOD) as the multiplexer 528 can allow the entire output of a single energy source 524, such as a single laser, to be directed into a plurality of individual energy guides 522A-522C. The guide beam 524B can be re-targeted to a different energy guide 522A-522C within microseconds by changing the driving frequency input into the multiplexer 528 (the AOD), and with a pulsed laser such as a Nd:YAG, this switching can easily occur between pulses. In such embodiments, the deflection angle (θ) of the multiplexer 528 can be defined as follows:
Deflection angle (θ)=Λf/v where,
Λ=Optical Wavelength
f=acoustic drive frequency
v=speed of sound in modulator

As shown in FIG. 5, the source beam 524A is directed from the energy source 524 toward the multiplexer 528, and is subsequently redirected due to the generated deflection angle as a desired guide beam 524B to each of the energy guides 522A-522C. More specifically, as illustrated, when the multiplexer 528 generates a first deflection angle for the source beam 524A, a first guide beam 524B1 is directed to the first energy guide 522A; when the multiplexer 528 generates a second deflection angle for the source beam 524A, a second guide beam 52462 is directed to the second energy guide 522B; and when the multiplexer 528 generates a third deflection angle for the source beam 524A, a third guide beam 52463 is directed to the third energy guide 522C. It is appreciated that, as illustrated, any desired deflection angle can include effectively no deflection angle at all, such that the guide beam 524B can be directed to continue along the same axial beam path as the source beam 524A.

In this embodiment, the multiplexer 528 (AOD) includes a transducer 572 and an absorber 574 that cooperate to generate the desired driving frequency that can, in turn, generate the desired deflection angle so that the source beam 524A is redirected as the desired guide beam 524B toward the desired energy guide 522A-522C. More particularly, the multiplexer 528 is configured to spatially control the source beam 524A. In the operation of the multiplexer 528, the power driving the acoustic transducer 572 is kept on, at a constant level, while the acoustic frequency is varied to deflect the source beam 524A to different angular positions that define the guide beams 524B1-524B3. Thus, the multiplexer 528 makes use of the acoustic frequency-dependent diffraction angle, such as described above.

FIG. 6 is a simplified schematic top view illustration of a portion of the catheter system 600 and still another embodiment of the multiplexer 628. More particularly, FIG. 6 illustrates a plurality of energy guides, such as a first energy guide 622A, a second energy guide 622B and a third energy guide 622C, an energy source 624, a system controller 626, and the multiplexer 628 that receives energy in the form of a source beam 624A, such as a single pulsed source beam, from the energy source 624 and selectively and/or alternatively directs the energy in the form of individual guide beams 624B in any desired sequence and/or pattern to any or all of the energy guides 622A-622C under control of the system controller 626. The energy guides 622A-622C, the energy source 624 and the system controller 626 are substantially similar in design and function as described in detail herein above. Accordingly, such components will not be described in detail in relation to the embodiment illustrated in FIG. 6. It is further appreciated that certain components of the system console 123 illustrated and described above in relation to FIG. 1, such as the power source 125 and the GUI 127, are not illustrated in FIG. 6 for purposes of simplicity and ease of illustration, but would typically be included in many embodiments.

It is appreciated that the multiplexer 628 illustrated in FIG. 6 is substantially similar to the multiplexer 528 illustrated and described in relation to FIG. 5. For example, as shown in FIG. 6, the multiplexer 628 again includes a transducer 672 and an absorber 674 that cooperate to generate the desired driving frequency that can, in turn, generate the desired deflection angle so that the source beam 624A is redirected as the desired guide beam 624B toward the desired energy guide 622A-622C. However, in this embodiment, the multiplexer 628 further includes an optical element 676 that is usable to transform the angular separation between the guide beams 624B into a linear offset.

In some embodiments, in order to improve the angular resolution and the efficiency of the catheter system 600, the input laser 624 should be collimated with a diameter close to filling the aperture of the multiplexer 628 (the AOD). The smaller the divergence of the input, the greater number of discrete outputs can be generated. The angular resolution of such a device is quite good, but the total angular deflection is limited. To allow a sufficient number of energy guides 622A-622C of finite size to be accessed by a single energy source 624 and a single source beam 624A, there are a number of means to improve the separation of the different output. For example, as shown in FIG. 6, after the individual guide beams 624B separate, the optical element 676, such as a lens, can be used to transform the angular separation between the guide beams 624B into a linear offset, and can be used to direct the guide beams 624B into closely spaced energy guides 622A-622C, such as when the energy guides 622A-622C are held in close proximity to one another within a guide coupling housing 650. Folding mirrors can be used to allow adequate propagation distance to separate the different beam paths of the guide beams 624B within a limited volume.

FIG. 7 is a simplified schematic top view illustration of a portion of the catheter system 700 and still yet another embodiment of the multiplexer 728. More particularly, FIG. 7 illustrates a plurality of energy guides, such a first energy guide 722A, a second energy guide 722B, a third energy guide 722C, a fourth energy guide 722D and a fifth energy guide 722E, an energy source 724, a system controller 726, and the multiplexer 728 that receives energy in the form of a source beam 724A, such as a single pulsed source beam, from the energy source 724 and selectively and/or alternatively directs the energy in the form of individual guide beams 724B in any desired sequence and/or pattern to any or all of the energy guides 722A-722E under control of the system controller 726. The energy guides 722A-722E, the energy source 724 and the system controller 726 are substantially similar in design and function as described in detail herein above. Accordingly, such components will not be described in detail in relation to the embodiment illustrated in FIG. 7. It is further appreciated that certain components of the system console 123 illustrated and described above in relation to FIG. 1, such as the power source 125 and the GUI 127, are not illustrated in FIG. 7 for purposes of simplicity and ease of illustration, but would typically be included in many embodiments.

It is appreciated that the manner for multiplexing the source beam 724A into multiple guide beams 724B illustrated in FIG. 7 is somewhat similar to how the source beam 524 was multiplexed into multiple guide beams 524B as illustrated and described in relation to FIG. 5. However, in this embodiment, the multiplexer 728 includes a pair of acousto-optic deflectors (AODs), i.e. a first acousto-optic deflector 728A and a second acousto-optic deflector 728B, that are positioned in series with one another. With such design, the multiplexer 728 may be able to access additional energy guides. It is further appreciated that the multiplexer 728 can include more than two acousto-optic deflectors, if desired, to be able to access even more energy guides.

In the embodiment shown in FIG. 7, the source beam 724A is initially directed toward the first AOD 728A. The first AOD 728A is utilized to deflect the source beam 724A to generate a first guide beam 724B1 that is directed toward the first energy guide 722A, and a second guide beam 724E32 that is directed toward the second energy guide 72262. The first AOD 728A also allows an undeviated beam to be transmitted through the first AOD 728A as a transmitted beam 724C that is directed toward the second AOD 728B. Subsequently, the second AOD 728B is utilized to deflect the transmitted beam 724C, as desired, to generate a third guide beam 72463 that is directed toward the third energy guide 722C, a fourth guide beam 724E34 that is directed toward the fourth energy guide 722D, and a fifth guide beam 72465 that is directed toward the fifth energy guide 72265.

Each AOD 728A, 728B can be designed in a similar manner to those described in greater detail above. For example, the first AOD 728A can include a first transducer 772A and a first absorber 774A that cooperate to generate the desired driving frequency that can, in turn, generate the desired deflection angle so that the source beam 724A is redirected as desired; and the second AOD 728B can include a second transducer 772B and a second absorber 774B that cooperate to generate the desired driving frequency that can, in turn, generate the desired deflection angle so that the transmitted beam 724C is redirected as desired. Alternatively, the first AOD 728A and/or the second AOD 728B can have another suitable design.

In various embodiments of the present invention, an optical pressure wave generator, such as a catheter system, designed to fracture vascular lesions 106A (illustrated in FIG. 1), such as calcified vascular lesions, requires multiple emitter stations 180 (illustrated in FIG. 1) distributed along its active length, within and/or relative to the length 142 (illustrated in FIG. 1) of the balloon 104 (illustrated in FIG. 1). Stated in another manner, the catheter system 100 (illustrated in FIG. 1) can include a plurality of emitter stations 180, with each emitter station 180 being positioned at a different longitudinal position relative to the length 142 of the balloon 104. For example, in one non-exclusive embodiment, the catheter system can include (i) a first emitter station 180 that is positioned at a first longitudinal position relative to the length 142 of the balloon 104, (ii) a second emitter station 180 that is positioned at a second longitudinal position relative to the length 142 of the balloon 104 that is different than the first longitudinal position, and (iii) a third emitter station 180 that is positioned at a third longitudinal position relative to the length 142 of the balloon 104 that is different than the first longitudinal position and the second longitudinal position. Each emitter station 180 incorporated within the single-use device can include a single emitter 135 (illustrated in FIG. 1), or multiple emitters 135, with each of the emitters 135 at any given emitter station 180 being located at approximately the same longitudinal position relative to the length 142 of the balloon 104. Stated in another manner, the guide distal end 122D (illustrated in FIG. 1) of an energy guide 122A (illustrated in FIG. 1) and the corresponding plasma generator 133 (illustrated in FIG. 1) that cooperate to form an individual emitter 135 within a particular emitter station 180, are located at approximately the same longitudinal position relative to the length 142 of the balloon 104 as the guide distal end 122D and the corresponding plasma generator 133 of any additional emitters 135 within that same emitter station 180.

The catheter system 100 can be configured to selectively provide power to multiple emitter stations 180 as part of a pressure wave-generating device that is designed to impart pressure onto and induce fractures in vascular lesions 106A, such as calcified vascular lesions and/or fibrous vascular lesions. In many embodiments, the catheter system 100 can be configured and controlled to selectively and/or separately power the multiple emitter stations 180 in any desired pattern, order, sequence, and rate of firing. Each emitter station 180 can also be configured to include any desired number of individual emitters 135, which can be a single emitter 135 or more than one emitter 135. In many embodiments, the catheter system 100 can be further configured and controlled to selectively and/or separately power each of the individual emitters 135 in any given emitter station 180 in any desired pattern, order, sequence, and rate of firing.

In many embodiments, it is desired that the emitter stations 180 are visible to the user or operator during use of the catheter system 100 in order that the user or operator can more precisely position the emitters 135 and/or emitter stations 180 relative to the vascular lesions 106A at the treatment site 106. Proper visibility of the emitters 135 and/or emitter stations 180 also enables the user or operator to selectively activate only those emitter stations 180 that are positioned most proximate to the vascular lesions 106A in order to more effectively and efficiently disrupt the vascular lesions 106A at the treatment site 106.

In various embodiments, the emitters 135 and/or the emitter stations 180 of the catheter system 100 can be formed from and/or can include a radiopaque material that is easily visible when used with fluoroscopy during an intravascular lithotripsy procedure. Alternatively, the emitters 135 and/or the emitter stations 180 can be formed from other suitable materials that can be made visible to the user or operator during an intravascular lithotripsy procedure.

FIG. 8 is a simplified schematic side view illustration of a portion of an embodiment of the catheter system 800 having features of the present invention. As illustrated, the catheter system 800 includes a balloon 804 having a balloon wall 830 that defines a balloon interior 846, and one or more emitter stations 880, such as a first emitter station 880A and a second emitter station 880B in this particular embodiment (although it is understood that the catheter system 800 can include any suitable number of emitter stations 880), that are positioned within the balloon interior 846 of the balloon 804. Each of the emitter stations 880A, 880B are positioned at different longitudinal locations relative to the length 842 of the balloon 804. Stated in another manner, as illustrated, the first emitter station 880A is positioned at a first longitudinal position 880L1 (or location) relative to the length 842 of the balloon 804, and the second emitter station 880B is positioned at a second longitudinal position 880L2 (or location) relative to the length 842 of the balloon 804 that is different than the first longitudinal position 880L1 (or location). It is appreciated that each of the emitter stations 880A, 880B can include any suitable number of emitters 135 (illustrated in FIG. 1), which can be one emitter 135 or multiple emitters 135. As such, each of the emitters 135 of any given emitter station 880 can be said to be positioned at approximately the same longitudinal position (or location) relative to the length 842 of the balloon 804.

During use of the catheter system 800, due to the visibility of the emitter stations 880 and/or emitters 135 as made possible through use of a radiopaque material or other suitable material for the emitter stations 880 and/or emitters 135, the user or operator can specifically select certain emitter stations 880 and/or emitters 135 to be used during an intravascular lithotripsy procedure, and/or can specifically deselect certain emitter stations 880 and/or emitters 135 that are not to be used during the intravascular lithotripsy procedure. It is appreciated that the specific selection or deselection of the emitter stations 880 and/or emitters 135 can be based at least in part on proximity to the vascular lesions 106A (illustrated in FIG. 1) at the treatment site 106 (illustrated in FIG. 1). For example, in some potential applications, the user or operator can select only one of the emitter stations 880 (and/or only one or more of the emitters 135 specifically included therein) to use during the intravascular lithotripsy procedure, such as only the first emitter station 880A or only the second emitter station 880B. Alternatively, in other potential applications, the user or operator can select both emitter stations 880A, 880B to use during the intravascular lithotripsy procedure.

FIG. 9 is a simplified schematic view illustration of a portion of another embodiment of the catheter system 900. In particular, in this embodiment, the catheter system 900 includes a balloon 904 having a balloon wall 930 that defines a balloon interior 946, and four emitter stations 980, such as a first emitter station 980A, a second emitter station 980B, a third emitter station 980C, and a fourth emitter station 980D, that are positioned within the balloon interior 946 of the balloon 904. Each of the emitter stations 980A, 980B, 980C, 980D are positioned at different longitudinal locations relative to the length 942 of the balloon 904. Stated in another manner, as illustrated, the first emitter station 980A is positioned at a first longitudinal position 980L1 (or location) relative to the length 942 of the balloon 904; the second emitter station 980B is positioned at a second longitudinal position 980L2 (or location) relative to the length 942 of the balloon 904 that is different than the first longitudinal position 980L1 (or location); the third emitter station 980C is positioned at a third longitudinal position 980L3 (or location) relative to the length 942 of the balloon 904 that is different than the first longitudinal position 980L1 (or location) and the second longitudinal position 980L2 (or location); and the fourth emitter station 980D is positioned at a fourth longitudinal position 980L4 (or location) relative to the length 942 of the balloon 904 that is different than the first longitudinal position 980L1 (or location), the second longitudinal position 980L2 (or location) and the third longitudinal position 980L3 (or location). It is appreciated that each of the emitter stations 980A, 980B, 980C, 980D can include any suitable number of emitters 135 (illustrated in FIG. 1), which can be one emitter 135 or multiple emitters 135. As such, each of the emitters 135 of any given emitter station 980 can be said to be positioned at approximately the same longitudinal position (or location) relative to the length 942 of the balloon 904.

During use of the catheter system 900, due to the visibility of the emitter stations 980 and/or emitters 135 as made possible through use of a radiopaque material or other suitable material for the emitter stations 980 and/or emitters 135, the user or operator can specifically select certain emitter stations 980 and/or emitters 135 to be used during an intravascular lithotripsy procedure, and/or can specifically deselect certain emitter stations 980 and/or emitters 135 that are not to be used during the intravascular lithotripsy procedure. It is appreciated that the specific selection or deselection of the emitter stations 980 and/or emitters 135 can be based at least in part on proximity to the vascular lesions 106A (illustrated in FIG. 1) at the treatment site 106 (illustrated in FIG. 1). For example, in some potential applications, the user or operator can select only one of the emitter stations 980 (and/or one or more of the emitters 135 included therein) to use during the intravascular lithotripsy procedure, such as only the first emitter station 980A, only the second emitter station 980B, only the third emitter station 980C, or only the fourth emitter station 980D. Alternatively, in other potential applications, the user or operator can select two emitter stations 980, such as (i) the first and second emitter stations 980A, 980B, (ii) the first and third emitter stations 980A, 980C, (iii) the first and fourth emitter stations 980A, 980D, (iv) the second and third emitter stations 980B, 980C, (v) the second and fourth emitter stations 980B, 980D, or (vi) the third and fourth emitter stations 980C, 980D, to use during the intravascular lithotripsy procedure. Still alternatively, in still other potential applications, the user or operator can select three emitter stations 980, such as (i) the first, second and third emitter stations 980A, 980B, 980C, (ii) the first, second and fourth emitter stations 980A, 980B, 980D, (iii) the first, third and fourth emitter stations 980A, 980C, 980D, or (iv) the second, third and fourth emitter stations 980B, 980C, 980D, to use during the intravascular lithotripsy procedure. Yet alternatively, in yet other potential applications, the user or operator can select all four emitter stations 980A, 980B, 980C, 980D to use during the intravascular lithotripsy procedure.

FIG. 10 is a simplified schematic view illustration of a portion of still another embodiment of the catheter system 1000. More particularly, in this embodiment, the catheter system 1000 includes a balloon 1004 having a balloon wall 1030 that defines a balloon interior 1046, and five emitter stations 1080, such as a first emitter station 1080A, a second emitter station 1080B, a third emitter station 1080C, a fourth emitter station 1080D, and a fifth emitter station 1080E, that are positioned within the balloon interior 1046 of the balloon 1004. Each of the emitter stations 1080A-1080E are positioned at different longitudinal locations relative to the length 1042 of the balloon 1004. Stated in another manner, as illustrated, the first emitter station 1080A is positioned at a first longitudinal position 1080L1 (or location) relative to the length 1042 of the balloon 1004; the second emitter station 1080B is positioned at a second longitudinal position 1080L2 (or location) relative to the length 1042 of the balloon 1004 that is different than the first longitudinal position 1080L1 (or location); the third emitter station 1080C is positioned at a third longitudinal position 1080L3 (or location) relative to the length 1042 of the balloon 1004 that is different than the first longitudinal position 1080L1 (or location) and the second longitudinal position 1080L2 (or location); the fourth emitter station 1080D is positioned at a fourth longitudinal position 1080L4 (or location) relative to the length 1042 of the balloon 1004 that is different than the first longitudinal position 1080L1 (or location), the second longitudinal position 1080L2 (or location) and the third longitudinal position 1080L3 (or location); and the fifth emitter station 1080E is positioned at a fifth longitudinal position 1080L5 (or location) relative to the length 1042 of the balloon 1004 that is different than the first longitudinal position 1080L1 (or location), the second longitudinal position 1080L2 (or location), the third longitudinal position 1080L3 (or location) and the fourth longitudinal position 1080L4 (or location). It is appreciated that each of the emitter stations 1080A-1080E can include any suitable number of emitters 135 (illustrated in FIG. 1), which can be one emitter 135 or multiple emitters 135. As such, each of the emitters 135 of any given emitter station 1080 can be said to be positioned at approximately the same longitudinal position (or location) relative to the length 1042 of the balloon 1004.

During use of the catheter system 1000, due to the visibility of the emitter stations 1080 and/or emitters 135 as made possible through use of a radiopaque material or other suitable material for the emitter stations 1080 and/or emitters 135, the user or operator can specifically select certain emitter stations 1080 and/or emitters 135 to be used during an intravascular lithotripsy procedure, and/or can specifically deselect certain emitter stations 1080 and/or emitters 135 that are not to be used during the intravascular lithotripsy procedure. It is appreciated that the specific selection or deselection of the emitter stations 1080 and/or emitters 135 can be based at least in part on proximity to the vascular lesions 106A (illustrated in FIG. 1) at the treatment site 106 (illustrated in FIG. 1). For example, in some potential applications, the user or operator can select only one of the emitter stations 1080 (and/or one or more of the emitters 13 included therein) to use during the intravascular lithotripsy procedure, such as only the first emitter station 1080A, only the second emitter station 10806, only the third emitter station 1080C, only the fourth emitter station 1080D, or only the fifth emitter station 1080E. Alternatively, in other potential applications, the user or operator can select two emitter stations 1080, such as (i) the first and second emitter stations 1080A, 1080B, (ii) the first and third emitter stations 1080A, 1080C, (iii) the first and fourth emitter stations 1080A, 1080D, (iv) the first and fifth emitter stations 1080A, 1080E, (v) the second and third emitter stations 1080B, 1080C, (vi) the second and fourth emitter stations 1080B, 1080D, (vii) the second and fifth emitter stations 1080B, 1080E, (viii) the third and fourth emitter stations 1080C, 1080D, (ix) the third and fifth emitter stations 1080C, 1080E, or (x) the fourth and fifth emitter stations 1080D, 1080E, to use during the intravascular lithotripsy procedure. Still alternatively, in still other potential applications, the user or operator can select three emitter stations 1080, such as (i) the first, second and third emitter stations 1080A, 1080B, 1080C, (ii) the first, second and fourth emitter stations 1080A, 1080B, 1080D, (iii) the first, second and fifth emitter stations 1080A, 1080B, 1080E, (iv) the first, third and fourth emitter stations 1080A, 1080C, 1080D, (v) the first, third and fifth emitter stations 1080A, 1080C, 1080E, (vi) the first, fourth and fifth emitter stations 1080A, 1080D, 1080E, (vii) the second, third and fourth emitter stations 1080B, 1080C, 1080D, (viii) the second, third and fifth emitter stations 1080B, 1080C, 1080E, (ix) the second, fourth and fifth emitter stations 1080B, 1080D, 1080E, or (x) the third, fourth and fifth emitter stations 1080C, 1080D, 1080E, to use during the intravascular lithotripsy procedure. Yet alternatively, in yet other potential applications, the user or operator can select four emitter stations 1080, such as (i) the first, second, third and fourth emitter stations 1080A, 1080B, 1080C, 1080D, (ii) the first, second, third and fifth emitter stations 1080A, 1080B, 1080C, 1080E, (iii) the first, second, fourth and fifth emitter stations 1080A, 1080B, 1080D, 1080E, (iv) the first, third, fourth and fifth emitter stations 1080A, 1080C, 1080D, 1080E, or (v) the second, third, fourth and fifth emitter stations 1080B, 1080C, 1080D, 1080E, to use during the intravascular lithotripsy procedure. Still yet alternatively, in still yet other potential applications, the user or operator can select all five emitter stations 1080A-1080E to use during the intravascular lithotripsy procedure.

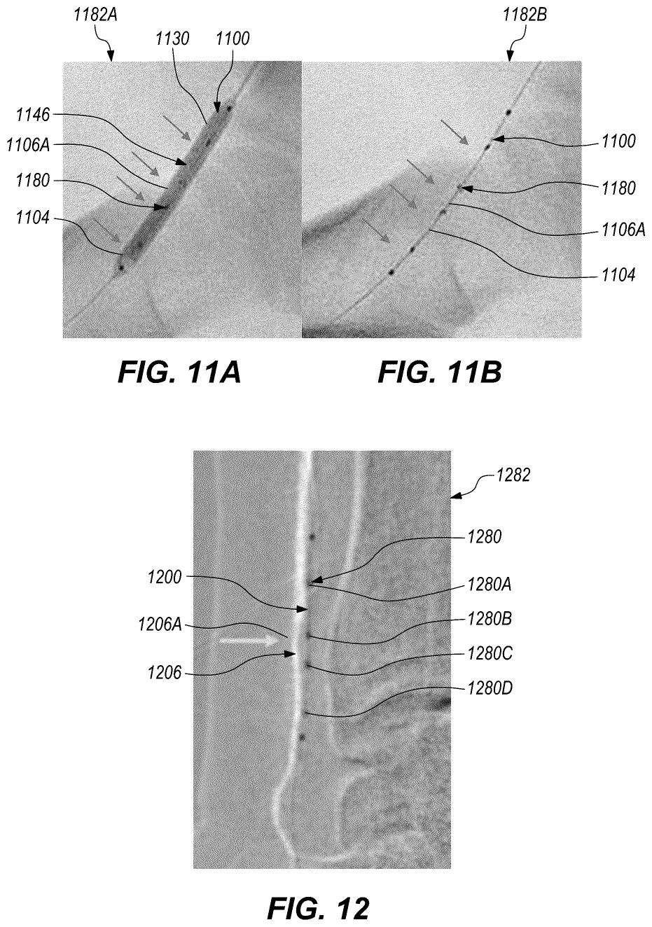

FIGS. 11A and 11B are fluoroscopic images of a portion of a catheter system 1100 that is positioned substantially adjacent to a vascular lesion 1106A. In particular, FIG. 11A is a fluoroscopic image 1182A of a portion of an embodiment of a catheter system 1100 that is positioned substantially adjacent to the vascular lesion 1106A, the catheter system 1100 including a balloon 1104 having a balloon wall 1130 that defines a balloon interior 1146 and four emitter stations 1180 that are positioned within the balloon interior 1146 of the balloon 1104, the balloon being in an inflated state; and FIG. 11B is a fluoroscopic image 1182B of the catheter system 1100 illustrated in FIG. 11A that is positioned substantially adjacent to the vascular lesion 1106A, the balloon 1104 being in a deflated state.

As shown in FIGS. 11A and 11B, the emitter stations 1180 of the catheter system 1100 are easily visible when used with fluoroscopy during an intravascular lithotripsy procedure due to the emitter stations 1180 being made from and/or including a radiopaque material.

Calcified lesions can come in all types of morphologies, ranging from short, focal lesions, to long lesions that are greater than 30 centimeters (cm) in length. Additionally, cross-sections of calcified lesions can be eccentric, nodular and circumferential. For any given lesion, the thickness of the calcium can range from a thin layer within the intimal section of the artery, to a thick layer that spans across the intimal and medial layers. In certain applications, a physician may not want to deliver energy to certain types of lesion morphologies. For instance, if a lesion is focal and only 10 millimeters (mm) in length, and is surrounded by a healthy, uncalcified artery, the physician may only want to target the calcified artery zone.

FIG. 12 is a fluoroscopic image 1282 of a portion of another embodiment of the catheter system 1200 that is positioned substantially adjacent to a vascular lesion 1206A at a treatment site 1206. In particular, FIG. 12 demonstrates a focal lesion with the catheter system 1200, an intravascular lithotripsy device, that has four emitter stations 1280, such as a first emitter station 1280A, a second emitter station 1280B, a third emitter station 1280C and a fourth emitter station 1280D, located inside of it.

As noted above, in various implementations, the user or operator can specifically select and/or deselect certain emitter stations 1280 based at least in part on proximity to the vascular lesions 1206A at the treatment site 1206. In the implementation illustrated in FIG. 12, the second emitter station 1280B and the third emitter station 1280C have been placed substantially adjacent to the vascular lesion 1206A at the treatment site 1206. In this situation, the user or operator may choose to turn on or select the second emitter station 1280B and the third emitter station 1280C so that energy is delivered to the vascular lesion 1206A, and/or turn off or deselect the first emitter station 1280A and the fourth emitter station 1280D so that energy is not delivered to healthy artery.

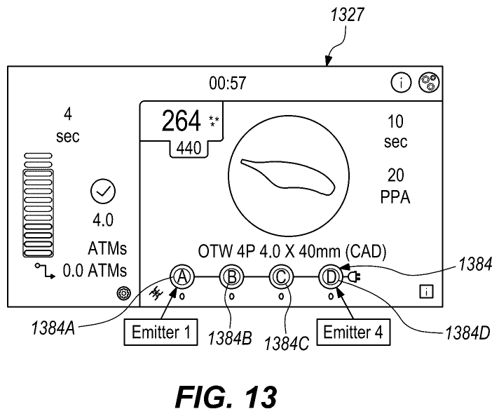

FIG. 13 is a simplified illustration of an embodiment of a graphical user interface 1327 that is usable as part of the catheter system. As shown in FIG. 13, the GUI 1327 includes four emitter activators 1384, such as a first emitter activator 1384A, a second emitter activator 1384B, a third emitter activator 1384C and a fourth emitter activator 1384D that correspond with the emitter stations 1280A-1280D shown in FIG. 12. In particular, the first emitter activator 1384A is usable to specifically activate (or select) or deactivate (deselect) the first emitter station 1280A (illustrated in FIG. 12); the second emitter activator 1384B is usable to specifically activate (or select) or deactivate (deselect) the second emitter station 1280B (illustrated in FIG. 12); the third emitter activator 1384C is usable to specifically activate (or select) or deactivate (deselect) the third emitter station 1280C (illustrated in FIG. 12); and the fourth emitter activator 1384D is usable to specifically activate (or select) or deactivate (deselect) the fourth emitter station 1280D (illustrated in FIG. 12).

In conjunction with the implementation illustrated in the image 1282 shown in FIG. 12, the GUI 1327 can include a touch screen display that can be utilized to specifically deselect, or turn off, the first emitter station 1280A and the fourth emitter station 1280D through corresponding emitter activators 1384A, 1384D. Additionally, or in the alternative, the touch screen display of the GUI 1327 can be utilized to specifically select, or turn on, the second emitter station 1280B and the third emitter station 1280C through corresponding emitter activators 1384B, 1384C. Thus, the user or operator is able to best target the vascular lesion 1206A at the treatment site 1206 while also best protecting the healthy, uncalcified portions of the artery.

As described in detail herein, in various embodiments, the present invention can be utilized to solve various problems that exist in more traditional catheter systems. For example, by enabling the catheter system to fire each emitter station and/or each emitter separately, it is possible to achieve a sequence or pattern of firing that could be much more effective and efficient for breaking localized lesions. Firing individual emitter stations and/or individual emitters in a desired sequenced pattern can more effectively break up a lesion at one particular location or an extended lesion.

In summary, based on the various embodiments of the present invention illustrated and described in detail herein, the catheter systems and related methods can include a catheter configured to advance to a vascular lesion, such as a calcified vascular lesion or a fibrous vascular lesion, at a treatment site located within or adjacent a blood vessel within a body of a patient. The catheter includes a catheter shaft, and an inflatable balloon that is coupled and/or secured to the catheter shaft. The balloon can include a balloon wall that defines a balloon interior. The balloon can be configured to receive a catheter fluid within the balloon interior to expand from a deflated state suitable for advancing the catheter through a patient's vasculature, to an inflated state suitable for anchoring the catheter in position relative to the treatment site.

In a pressure wave-generating medical device, such as the catheter systems as described herein, it is often desirable to have a number of potential output channels, or emitter stations (or emitters), for the treatment process.

In certain embodiments, the catheter systems and related methods utilize an energy source which provides energy that is guided by one or more energy guides disposed along the catheter shaft and within the balloon interior of the balloon to create a localized plasma in the catheter fluid that is retained within the balloon interior of the balloon at or near a guide distal end of each of the energy guides disposed within the balloon interior of the balloon that is located at the treatment site. The creation of the localized plasma can initiate a pressure wave and can initiate the rapid formation of one or more bubbles that can rapidly expand to a maximum size and then dissipate through a cavitation event that can launch a pressure wave upon collapse. The rapid expansion of the plasma-induced bubbles can generate one or more pressure waves within the catheter fluid retained within the balloon interior of the balloon and thereby impart pressure waves onto and induce fractures in the vascular lesions at the treatment site within or adjacent to the blood vessel wall within the body of the patient.

The guide distal end of each of the plurality of energy guides can be positioned in any suitable locations relative to a length of the balloon to more effectively and precisely impart pressure waves for purposes of disrupting the vascular lesions at the treatment site.

Each energy guide can be used in conjunction with a corresponding plasma generator that is positioned at or near the guide distal end of the energy guide, and spaced apart from the guide distal end of the energy guide in many embodiments, within the balloon interior of the balloon located at the treatment site for creating the localized plasma and/or for generating the desired pressure waves within the balloon interior for purposes of disrupting the vascular lesions. As utilized herein, the guide distal end of the energy guide and the corresponding plasma generator can be referred to collectively as an “emitter”. In some applications, one or more emitters that are positioned at approximately the same longitudinal position within the balloon interior of the balloon can be referred to as an “emitter station”.

Thus, the catheter systems and related methods disclosed herein are configured to provide a means to power multiple emitter stations and/or multiple emitters in a pressure wave-generating device that is designed to impart pressure onto and induce fractures in vascular lesions. In many embodiments, the catheter systems can be configured and controlled to selectively and/or separately power the multiple emitter stations in any desired pattern, order, sequence, and rate of firing.

Importantly, in various embodiments, the emitter stations and/or the emitters of the catheter system can be formed from and/or can include a radiopaque material that is easily visible when used with fluoroscopy during an intravascular lithotripsy procedure. Thus, the visibility of the emitter stations and/or the emitters enables the user or operator to more precisely position the emitter stations and/or the emitters as desired substantially adjacent to the vascular lesions, and/or to selectively activate only those emitter stations and/or emitters that are positioned most proximate to the vascular lesions in order to more effectively and efficiently disrupt the vascular lesions.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the detailed description provided herein. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter systems have been illustrated and described herein, one or more features of any one embodiment can be combined with one or more features of one or more of the other embodiments, provided that such combination satisfies the intent of the present invention.

While a number of exemplary aspects and embodiments of the catheter systems have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope, and no limitations are intended to the details of construction or design herein shown.

1. A catheter system for treating a treatment site within or adjacent to a vessel wall, the catheter system comprising:
an energy source that generates energy;
a plurality of energy guides that are each configured to selectively receive the energy from the energy source, each of the plurality of energy guides including a corresponding guide distal end, the energy that is received by each of the plurality of energy guides being emitted from the corresponding guide distal end; and
a plurality of emitters that are each positionable near the treatment site, each emitter including the corresponding guide distal end of one of the plurality of energy guides, at least one of the emitters including a radiopaque material.

2. The catheter system of claim 1 wherein the radiopaque material is visible when used with fluoroscopy during use of the catheter system in an intravascular lithotripsy procedure.

3. The catheter system of claim 1 further comprising a catheter shaft and a balloon that is coupled to the catheter shaft, the balloon including a balloon wall that defines a balloon interior, the balloon being configured to retain a catheter fluid within the balloon interior; wherein the energy guides are disposed along the catheter shaft, and the corresponding guide distal end of each of the energy guides is positioned within the balloon interior so that each of the emitters is positioned within the balloon interior.

4. The catheter system of claim 3 wherein each emitter further includes a corresponding plasma generator that is positioned near the corresponding guide distal end of the one of the plurality of energy guides, the energy that is received by each of the plurality of energy guides being emitted from the corresponding guide distal end and impinging on the corresponding plasma generator so that plasma is generated in the catheter fluid retained within the balloon interior.

5. The catheter system of claim 4 wherein the plasma generation causes bubble formation that generates a pressure wave that imparts pressure adjacent to the vessel wall.

6. The catheter system of claim 3 further comprising further comprising a plurality of emitter stations that are positioned within the balloon interior, each emitter station being positioned at a different longitudinal position within the balloon interior relative to a length of the balloon than each of the other emitter stations, each emitter station including at least one of the plurality of emitters; and wherein at least one of the plurality of emitter stations includes a radiopaque material.

7. The catheter system of claim 6 wherein each of the plurality of emitter stations includes a radiopaque material that is visible when used with fluoroscopy during use of the catheter system in an intravascular lithotripsy procedure.

8. The catheter system of claim 6 wherein the plurality of emitter stations includes a first emitter station including a first plurality of emitters that are each positioned at a first longitudinal position within the balloon interior, and a second emitter station that includes a second plurality of emitters that are each positioned at a second longitudinal position within the balloon interior that is different than the first longitudinal position.

9. The catheter system of claim 1 further comprising a system controller including a processor that controls the energy source so that the energy from the energy source is selectively directed to each of the emitters in any desired pattern of firing; and wherein the system controller is configured to one of specifically select and specifically deselect the emitters to be activated during use of the catheter system in an intravascular lithotripsy procedure based at least in part on proximity of the emitters to the treatment site.

10. The catheter system of claim 9 further comprising a graphical user interface that includes a plurality of emitter activators that one of (i) specifically selects and (ii) specifically deselects the emitters to be activated during use of the catheter system in the intravascular lithotripsy procedure.

11. The catheter system of claim 1 further comprising a multiplexer that receives the energy from the energy source and directs the energy from the energy source in the form of individual guide beams to each of the plurality of energy guides.

12. The catheter system of claim 1 wherein the energy source is a light source that generates pulses of light energy.

13. The catheter system of claim 12 wherein the light source is a laser.

14. The catheter system of claim 1 wherein each of the plurality of energy guides includes an optical fiber.

15. A method for treating a treatment site within or adjacent to a vessel wall, the method comprising the steps of:
generating energy with an energy source;
selectively receiving the energy from the energy source with each of a plurality of energy guides, each of the plurality of energy guides including a corresponding guide distal end, the energy that is received by each of the plurality of energy guides being emitted from the corresponding guide distal end; and
positioning a plurality of emitters near the treatment site, each emitter including the corresponding guide distal end of one of the plurality of energy guides, at least one of the emitters including a radiopaque material.

16. The method of claim 15 further comprising the steps of coupling a balloon to a catheter shaft, the balloon including a balloon wall that defines a balloon interior; and receiving a catheter fluid within the balloon interior; wherein the step of selectively receiving includes disposing the energy guides along the catheter shaft; wherein the step of positioning includes positioning the corresponding guide distal end of each of the energy guides within the balloon interior so that each of the emitters is positioned within the balloon interior, and positioning a corresponding plasma generator of each of the emitters near the corresponding guide distal end of one of the plurality of energy guides; and wherein the energy that is received by each of the plurality of energy guides is emitted from the corresponding guide distal end and impinges on the corresponding plasma generator so that plasma is generated in the catheter fluid retained within the balloon interior.

17. The method of claim 16 further comprising the step of positioning a plurality of emitter stations within the balloon interior, the plurality of emitter stations including a first emitter station including a first plurality of emitters that are each positioned at a first longitudinal position within the balloon interior, and a second emitter station that includes a second plurality of emitters that are each positioned at a second longitudinal position within the balloon interior that is different than the first longitudinal position, at least one of the plurality of emitter stations including a radiopaque material that is visible when used with fluoroscopy during an intravascular lithotripsy procedure.

18. The method of claim 15 further comprising the step of controlling the energy source with a system controller including a processor so that the energy from the energy source is selectively directed to each of the emitters in any desired pattern of firing; and wherein the system controller is configured to one of specifically select and specifically deselect the emitters to be activated during an intravascular lithotripsy procedure based at least in part on proximity of the emitters to the treatment site.

19. The method of claim 15 further comprising the steps of receiving the energy from the energy source with a multiplexer, and directing the energy from the energy source in the form of individual guide beams to each of the plurality of energy guides with the multiplexer.

20. The method of claim 15 wherein the step of generating includes the energy source being a light source that generates pulses of light energy; and wherein the step of selectively receiving includes each of the plurality of energy guides including an optical fiber.

21. The catheter system of claim 1 further comprising a graphical user interface that is configured to allow a user to one of (i) activate, and (ii) deactivate any of the plurality of emitters.

22. The method of claim 15 further comprising the step of providing a graphical user interface that allows a user to one of (i) activate, and (ii) deactivate any of the plurality of emitters.
